(19) United States
(12) Patent Application Publication
(10) Pub. No.: US 2023/0410942 A1
(43) Pub. Date: Dec. 21, 2023

(54) USE OF NON-ERROR-PROPAGATING PHASING TECHNIQUES AND COMBINATION OF ALLELIC BALANCE TO IMPROVE CNV DETECTION

(71) Applicant: MYOME, INC., MENLO PARK, CA (US)

(72) Inventors: AKASH KUMAR, PALO ALTO, CA (US); MATTHEW RABINOWITZ, SAN FRANCISCO, CA (US)

(73) Assignee: MYOME, INC., MENLO PARK, CA (US)

(21) Appl. No.: 18/251,096

(22) PCT Filed: Oct. 29, 2021
(86) PCT No.: PCT/US2021/057400

§ 371 Date: Apr. 28, 2023

Related U.S. Application Data
(60) Provisional application No. 63/107,464, filed on Oct. 30, 2020.

(51) Int. Cl. G16B 20/10; G16B 30/10; C12Q 1/6869

(52) U.S. Cl. CPC G16B 20/10; G16B 30/10; C12Q 1/6869

(57) ABSTRACT

Disclosed herein are methods of using non-error-propagating phasing techniques in combination with sequencing data obtained through more conventional error-propagating approaches to improve phasing of a genome and correct allele balance signals, which may allow for improved determinations of ploidy status of chromosomal segments. Further disclosed herein are methods of using allele balance and depth of read in combination to make improved ploidy status determinations. The techniques described herein may be used in a minimally invasive manner to make ploidy status determinations for an embryo or fetus and to identify chromosomal instability in tumor DNA.

### CROSS-REFERENCE TO RELATED APPLICATIONS

This application claims the benefit of U.S. Provisional Application No. 63/107,464 filed on Oct. 30, 2020, which is herein incorporated by reference in its entirety.

### BACKGROUND

Copy number variants (CNVs) can be important indicators of disease and disease progression. CNVs have been identified as a major cause of structural variation in the genome, involving both duplications and deletions of sequences that typically range in length from 1 kb to 20 Mb. Deletions and duplications of chromosome segments or entire chromosomes are associated with a variety of conditions, such as susceptibility or resistance to disease. However, methods of identifying CNVs remains challenging and is complicated by multiple issues. In some instances, normal tissue and abnormal tissue (comprising one or more CNVs) are mixed together, creating noise which hinders the detection of the one or more CNVs. Also, the sequencing data available may have limited dynamic range. Additionally, uneven amplification due to resampling bias may result in skewed variant allele balance.

Thus, improved methods are needed to more accurately detect deletions and duplications of chromosome segments or entire chromosomes, including CNVs. Preferably, these methods can be used to more accurately diagnose disease or an increased risk of disease, such as cancer or CNVs in a gestating fetus.

### SUMMARY

According to one aspect of the invention, disclosed herein is a method of correcting an allele balance signal for a chromosomal segment. The method involves obtaining a reference genetic code, which may be at least partially phased, that has at least two phase sets. Each phase set has one or more variants of interest. The method further involves obtaining the allele balance signal for the one or more variants of interest from sequencing performed on a sample of genetic material, and obtaining a plurality of reads sequenced using a non-error-propagating technique. Each read covers at least one of the one or more variants of interest. The phase alignment of the two phase sets is then determined as being in phase or out of phase based on the plurality of reads, and a true allele balance signal is determined by confirming, correcting, or supplying the phasing of at least one variant of interest based on the determined phase alignment of the two phase sets.

The non-error-propagating technique may involve conformation capture, single-cell template strand sequencing, or chromosomal isolation (e.g., via laser capture microdissection or karyotype). The method may entail performing the non-error-propagating technique to obtain the plurality of reads. The method may entail performing the sequencing on the sample of genetic material to obtain the allele balance signal.

The allele balance signal and the plurality of reads may be derived from the same sample of genetic material. The sample may be a body fluid sample (e.g., a blood sample, a saliva sample) or a tissue biopsy sample. The allele balance signal and the plurality of reads may be derived from a same population of cells. The allele balance signal may be derived from cell-free DNA and the plurality of reads derived from cellular DNA. The cellular DNA may be from cells found within a body fluid (e.g., blood or saliva).

The reference genetic code may be derived from the sequencing used to generate the allele balance signal. The reference genetic code may be derived, at least in part, from sequencing of normal tissue in a subject for which the allele balance signal is obtained; from sequencing of germline tissue in the subject; or from sequencing genetic material from one or more genetic relatives of the subject. The one or more relatives may be the subject's mother and/or a father. The reference genetic code may be derived, at least in part, from germline sequencing of the one or genetic relatives.

The reference genetic code may be derived, at least in part, from whole genome shotgun sequencing of the subject. The allele balance signal may be derived from the whole genome shotgun sequencing. In either case, the whole genome shotgun sequencing may be performed on cell-free DNA in a body fluid sample (e.g., a blood sample or saliva sample). The non-error-propagating technique may entail single cell sequencing. The method may further entail collecting a sample of genetic material from which the allele balance signal is derived and/or collecting a sample of genetic material from which the plurality of reads are derived.

Correcting the allele balance data may entail correcting a switch error in a reference genetic code, which has been at least partially phased. The allele balance signal may be averaged over a plurality of binned variants within a region of about, at least about, or no greater than about 50,000, 100,000, 200,000, 300,000, 400,000, 500,000, 750,000, 1,000,000, 50,000,000, or 100,000,000 bp. The allele balance may be averaged over one or more haplotype blocks. The one or more haplotype blocks may have been determined by dilution pool sequencing. The allele balance signal may have been derived from the same sequencing used to determine the one or more haplotype blocks. The allele balance signal may be filtered for a minimum read depth, such as, for example, a minimum read depth of 5, 10, 15, 20, or 25 reads.

The two phase sets may be neighboring phase sets within the reference genetic code. For instance, each of the neighboring phase sets may encompass a variant of interest which is no further than about 1,000, 5,000, 10,000, 50,000, 100,000, 500,0000, 1,000,000, 5,000,000, 10,000,000, 100,000,000, or 250,000,000 bp from a variant of interest in the other. The plurality of reads may be filtered for reads comprising at least 2, 3, 4, or 5 of the variants of interest from each of the two phase sets.

The non-error-propagating technique may entail chromosome conformation capture, specifically. The chromosome conformation capture technique may be Hi-C. Determining the phase alignment based on the plurality of reads may entail determining whether most of the reads are concordant or discordant with respect to a presumed phasing alignment between the two phase sets, which may be based on an at least partial phasing of the reference genetic code. Determining the phase alignment based on the plurality of reads may entail determining or estimating a probability that an amount of concordance or discordance observed between the two phase sets from the plurality of reads is the result of chance. The probability may be a binomial probability, optionally assuming that there is an equal chance than an observed fragment will be concordant or discordant.

The method may further entail using the corrected allele balance signal to determine a ploidy status for a chromosomal segment. For example, determining the ploidy status may be calling a copy number variant (CNV).

According to another aspect of the invention, disclosed herein is a method of determining a ploidy status for a chromosomal segment. The method involves obtaining a depth of read signal for a first set of one or more variants within the chromosomal segment; obtaining an allele balance signal for a second set of one or more variants within the chromosomal segment; and using the depth of read signal in combination with the allele balance signal to determine the ploidy status of the chromosomal segment.

Determining the ploidy status of the chromosomal segment may entail determining whether or not a CNV exists within the chromosomal segment. Obtaining the depth of read signal may entail obtaining a number of sequencing reads mapped to at least one of the variants within the first set normalized relative to a total number of reads. The depth of read signal and/or the allele balance signal may be averaged over a binned plurality of variants within a region of about, at least about, or no greater than about 50,000, 100,000, 200,000, 300,000, 400,000, 500,000, 750,000, 1,000,000, 50,000,000, or 100,000,000 bp. The depth of read signal and/or allele balance signal may be averaged over one or more haplotype blocks. The one or more haplotype blocks may have been determined by dilution pool sequencing. The depth of read signal and the allele balance signal may be averaged over the same binned region.

Using the depth of read signal in combination with the allele balance signal may entail making a positive or negative determination only when both the depth of read signal exceeds a depth of read threshold and the allele balance signal exceeds an allele balance threshold or when neither the depth of read signal exceeds the depth of read threshold nor the allele balance signal exceeds the allele balance threshold. Using the depth of read signal in combination with the allele balance signal may entail combining the depth of read signal and the allele balance signal into a single combined signal. Combining the depth of read signal and the allele balance signal into a single combined signal may involve multiplying the signals together or adding the signals together. The combined signal may be averaged over a binned plurality of variants within a region of about, at least about, or no greater than about 50,000, 100,000, 200,000, 300,000, 400,000, 500,000, 750,000, 1,000,000, 50,000,000, or 100,000,000 bp. The combined signal may be averaged over one or more haplotype blocks, which may have been determined by dilution pool sequencing. The combined signal may be averaged over a plurality of bins across which the depth of read signal and/or the allele balance signal were averaged.

The first set of one or more variants may consist of only 1 variant. The first set of one or more variants may have at least 2, 3, 4, 5, 10, 20, 30, 40, 50, 60, 70, 80, 90, 100, 200, 300, 400, 500, 600, 700, 800, 900, or 1,000 variants. The second set of one or more variants consists of only 1 variant. The second set of one or more variants may have at least 2, 3, 4, 5, 10, 20, 30, 40, 50, 70, 80, 90, 100, 200, 300, 400, 500, 600, 700, 800, 900, or 1,000 variants. The first set of one or more variants may be identical to the second set of one or more variants.

Obtaining the depth of read signal and/or obtaining the allele balance signal may entail performing sequencing. The depth of read signal and allele balance signal may be derived from the same sequencing data. The depth of read signal and/or the allele balance signal may be filtered for a minimum read depth, such as, for example, a minimum read depth of 5, 10, 15, 20, or 25 reads.

The method may entail calculating an individual probability of accurate determination of ploidy status based on the depth of read signal and/or the allele balance signal or calculating a joint probability of accurate determination of ploidy status based on the depth of read signal and the allele balance signal. The probabilities, for example, may measure the probability of one of the following: a true positive, a false positive, a true negative, and a false negative. At least one of the following may be determined to be true: the joint probability of a false positive is less than both of the individual probabilities of a false positive; the joint probability of a false negative is less than both of the individual probabilities of a false negative; the joint probability of a true positive is greater than both of the individual probabilities of a true positive; or the joint probability of a true negative is greater than both of the individual probabilities of a true negative.

The depth of read signal may be offset against a first baseline signal and/or the allele balance signal may be offset against a second baseline signal. Each baseline signal may be based on a mean signal for a second chromosomal segment having a known ploidy status. The second chromosomal segment may be within the same chromosome as the chromosomal segment for which the ploidy status is being determined. The depth of read signal and/or the allele balance signal may be normalized against a measure of noise within the signal. The measure of noise may be the standard deviation or variance of the signal over the chromosomal segment for which the ploidy status is being determined, over the second chromosomal segment having a known ploidy status, over a third chromosomal segment having a known ploidy status of interest that is different from the ploidy status of the second chromosomal segment, or over the entire chromosome. The variance in the depth of read signal and a variance within the allele balance signal may be within 100, 90, 80, 70, 60, 50, 40, 30, 20, 10, 9, 8, 7, 6, 5, 4, 3, 2, 1.9, 1.8, 1.7, 1.6, 1.5, 1.4, 1.3, 1.2, or 1.1 fold of each other. Using the depth of read signal in combination with the allele balance signal may result in reducing the false positive rate and/or the false negative rate by at least about 2, 3, 4, 5, 6, 7, 8, 9, 10, 20, 30, 40, 50, 60 70, 80, 90, 100, 150, 200, 250, or 500 fold relative to the false positive rate and/or false negative rate obtained with using one or both of the signals individually.

Using the depth of read signal in combination with the allele balance signal may involve selecting a depth of read threshold and an allele balance threshold. The signal thresholds may each be calculated as half the mean value of the respective signal averaged over a plurality of variants known to exhibit a ploidy status of interest (e.g., an aneuploidy). Using the depth of read signal in combination with the allele balance signal may involve selecting a combined signal threshold. The combined signal threshold may be calculated as half the mean value of a combined signal averaged over a plurality of variants known to exhibit a ploidy status of interest (e.g., an aneuploidy).

The method may result in an aneuploidy of one or more chromosomes being detected. The method may result in euploidy of all chromosomes analyzed being detected. The method may result in an addition and/or deletion of a chromosomal segment being detected. The method results in a CNV being identified.

Obtaining the allele balance signal may entail correcting an original allele balance signal by performing any one of the aforementioned methods for doing so that are described elsewhere herein.

According to another aspect of the invention, any of the aforementioned methods may entail obtaining a signal indicative of ploidy status (e.g., the allele balance signal or depth of read signal) that is derived from a sample comprising a population of cells having different copy numbers for the chromosomal segment. Some of the cells within the population of cells may have an aneuploidy while others may not. The signal may be derived from a sample comprising one or more tumor cells. The sample may further include non-tumor cells.

According to another aspect of the invention, any of the aforementioned methods may entail obtaining a signal indicative of ploidy status (e.g., the allele balance signal or depth of read signal) that is derived from cell-free DNA. The cell-free DNA may be cell-free fetal DNA (cffDNA) or circulating tumor DNA (ctDNA).

According to another aspect of the invention, any of the aforementioned methods may entail obtaining a signal indicative of ploidy status (e.g., the allele balance signal or depth of read signal) that from an embryo or a fetus. The embryo may be an embryo existing in vitro, such as, for example, prior to implantation of the embryo into a womb.

According to another aspect of the invention, disclosed herein is a method of detecting chromosomal instability in tumor DNA. The method involves determining a ploidy status according to any one of aforementioned methods for doing so for one or more chromosomal segments within a sample of genetic material. The sample of genetic material is at least partially derived from DNA originating from one or more cells known to be or suspected to be tumor cells. Identification of an aneuploidy status for the one or more chromosomal segments is used to indicate chromosomal instability of at least some tumor cells.

The sample may be from a subject diagnosed with or suspected of having cancer. The sample may contain circulating tumor DNA. Sequencing of normal tissue (e.g., germline tissue) or tumor tissue from a subject from which the genetic material is obtained may be used to establish a reference genetic code. The method may further entail treating the one or more cells or a subject from which the genetic material is obtained for cancer based on whether chromosomal instability has been indicated. The treatment may involve administering poly ADP ribose polymerase (PARP) inhibitors and/or platinum-based chemotherapeutics to the one or more cells or subject if chromosomal instability is indicated.

According to another aspect of the invention, disclose herein is a method of detecting a de novo copy number variant (CNV) in a subject. The method involves determining a ploidy status according to any one of the aforementioned methods for doing so for a chromosomal segment. The parents of the subject are euploid for the chromosomal segment. A de novo aneuploid (e.g., CNV) may be identified in the chromosomal segment of the subject by performing the method.

The determination of ploidy status may entail comparing the ploidy status to a reference genetic code derived from sequencing performed on one or more genetic relatives of the subject. The one or more genetic relatives may be the subject's mother and/or a father. The sequencing may be performed with a non-error-propagating technique to provide a plurality of reads according to any one of the aforementioned methods for doing so. The sequencing may be performed on cellular DNA. The method may further entail determining whether the mother or father of the subject is the source of an aneuploidy.

The subject may be an embryo. The method may entail obtaining a signal indicative of ploidy status (e.g., the allele balance signal or depth of read signal) that is derived from an embryo biopsy, blastocele fluid, or cell culture medium (cell-free DNA in the culture medium). The method may further entail selecting the embryo based on the absence or presence of an aneuploidy. The embryo may be selected from a plurality of embryos. The selected embryo may be used for in vitro fertilization (IVF), may be disposed of, or may be frozen.

The subject may be a fetus. The method may entail obtaining a signal indicative of ploidy status (e.g., the allele balance signal or depth of read signal) that is derived from cell-free fetal DNA (cffDNA). The method may entail treating the fetus and/or the mother based on the identified absence or presence of an aneuploidy (e.g., CNV). The treatment may entail performing additional testing on the fetus, such as, for example, karyotyping. The treatment may entail terminating a pregnancy. The treatment may entail administering a prenatal treatment to the fetus for a disease associated with the presence of a detected aneuploidy (e.g., CNV).

According to another aspect of the invention, disclosed herein is a method of screening a subject for a disease. The method involves determining whether one or more genetic variants associated with the disease is present. The one or more genetic variants include an aneuploidy (e.g., CNV) that was identified by performing by any one of the aforementioned methods for determining ploidy status on one or more other subjects and/or an SNP that was present within a same haplotype block as the aneuploidy. The SNP may be known to be associated with the disease.

The CNV and SNP may be in linkage disequilibrium. Determining whether the one or more genetic variants associated with the disease is present may involve performing sequencing on the subject. A portion of the genome encompassing the one or more genetic variants may be targeted for sequencing (e.g., via a microarray). The method may entail calculating a polygenic risk score (PRS) for the disease based at least in part on the one or more genetic variants. The method may further entail diagnosing the subject with a disease based, at least in part, on the presence or absence of the one or more genetic variants or on a PRS based, at least in part, on the one or more genetic variants. The method may entail treating the subject based on the presence or absence of the one or more genetic variants.

According to another aspect of the invention, disclosed herein is a method of phasing a germline mosaic variant in a subject. The method involves obtaining a reference genetic code having at least two phase sets. Each phase set has one or more variants of interest. The reference genetic code may be at least partially phased. The method further involves obtaining a plurality of reads sequenced using a non-error-propagating technique. Each read comprises at least one of the one or more variants of interest. The phase alignment of the two phase sets are determined as being in phase or out of phase based on the plurality of reads, and a haplotype encompassing a chromosomal segment exhibiting an aneuploidy (e.g., CNV) is identified based on the determined phase alignment of the two phase sets.

The subject may be diagnosed or suspected as having a genetic disease or condition associated with the aneuploidy. The subject may have been diagnosed as having or may be suspected of having Noonan Syndrome or RASopathy. The method may further entail screening gametes from the subject for the identified haplotype. The method may further entail selecting a gamete not having the identified haplotype for in vitro fertilization. The method may entail screening for the haplotype in an embryo during preimplantation genetic testing. The method may entail selecting an embryo based on the absence or presence of the aneuploidy. The embryo may be selected from a plurality of embryos. The method may entail using the selected embryo in in vitro fertilization (IVF), disposing of the selected embryo, or freezing the selected embryo. The aneuploidy may be identified by performing the method of any one of the aforementioned methods for determining ploidy status.

### BRIEF DESCRIPTION OF THE DRAWINGS

FIG. 1 depicts the simulated allele balance data for human chromosome 21 having an amplification approximately between nucleotide positions 30.2 Mb and 44.3 Mb.

FIG. 2 depicts the simulated allele balance data when averaged over haplotype blocks. The arrow depicts the approximate location of the switch error in the inputted phased genotype data which causes the appearance of a monosomy rather than a trisomy downstream of the switch error as actually simulated in the chromosome.

FIG. 3 depicts the simulated allele balance data when averaged over 300 Kb windows of the haplotype blocks, which are depicted in the lower part of the figure over the region of the chromosome where an aneuploidy is detected.

FIG. 4 depicts a summary of the Hi-C data for the genetic sample from which the allele balance data was simulated.

FIG. 5 depicts the true allele balance signal after the switch error is corrected.

FIGS. 6A-6B depict the simulated true allele balance signal for a scenario comprising a mixture of chromosomes comprising the normal disomic region and the abnormal trisomic region.

FIG. 6A shows the signal for individual measurements, and FIG. 6B shows the signal when averaged over haplotype blocks.

FIG. 7 schematically illustrates a population of disomic measurements and a population of trisomic measurements (shaded) as normal distributions spread across two different signals, X₁ and X₂, wherein m₁ and m₂ refer to mean measurements for the trisomic populations (trisomic regions of the chromosome).

FIGS. 8A-8B depict depth of read data for the region of the chromosome having the simulated amplification. FIG. 8A depicts the raw depth signal for each indexed position, and FIG. 8B depicts a histogram showing the proportion of measurements for various binned depths of read.

FIGS. 9A-9C depict allele balance data for the region of the chromosome having the simulated amplification. FIG. 9A depicts the raw allele balance signal for each indexed position, and FIG. 9B depicts a histogram showing the frequency of measurements for various binned proportions of the A allele. FIG. 9C further depicts the histogram where the measurements were averaged across 50 neighboring SNPs.

FIG. 10 depicts the depth of read signal across the simulated amplification (trisomy) between positions 30 Mb and 37 Mb, offset against the disomy depth of read signal and normalized for the noise (standard deviation) of the trisomy depth of read signal.

FIG. 11 depicts the allele balance signal across the simulated amplification (trisomy) between positions 30 Mb and 37 Mb, offset against the disomy allele balance signal and normalized for the noise (standard deviation) of the trisomy allele balance signal.

FIG. 12 depicts the combination of the offset and normalized depth of read and allele balance signals by addition.

### DETAILED DESCRIPTION

Disclosed herein are methods of making improved determinations of ploidy status by applying nucleotide sequencing methodologies that are non-error-propagating in nature to phase one or more regions of a genetic code of interest (e.g., a genome of interest), particularly regions that may contain a switch error introduced from previous error-propagating phasing techniques. The phase alignment determined between two or more variants of interest via the non-error-propagating methodology may be combined with existing phase information for the genetic code of interest. In some instances, the determined phase alignment may be used to correct the phasing of one or more variants of interest which were incorrectly phased (e.g., from a phasing technique that introduced a switch error). In some instances, the determined phase alignment may be used to confirm the presumed phasing of one or more variants is the true phasing. In some instances, the determined phase alignment may be used to supply missing phase information. The phasing information for a portion of the genetic code of interest, determined at least in part by the non-error propagating methodology, may be used to (re)analyze an allele balance signal. The true allele balance signal obtained from using the non-error-propagating phasing methodologies may be used to make improved determinations of ploidy status, such as CNV calls. In particular implementations, the improved phasing alignment may be used to determine whether an allele balance signal indicative of a shift in allele balance relative to a reference haplotype corresponds to a deletion or amplification within the genetic code of interest.

Also disclosed herein are methods of making improved determinations of ploidy status by using an allele balance signal in combination with a depth of read signal. Such signals provide orthogonal information that can improve the signal-to-noise ratio and reduce the probability of false positive and/or false negative calls. The use in combination may be particularly powerful where the allele balance signal is corrected via non-error-propagating phasing approaches to provide a true allele balance signal.

### Phasing and Switch Errors

Switch errors occur when a variant location is incorrectly phased with respect to its neighboring variants. As used herein, a “variant” may refer to any difference between the sequence of two or more homologous chromosomes, including single nucleotide polymorphisms (SNPs). As used herein, variants carry no implication of sufficiently low frequency in a larger population, unless indicated otherwise by context. Phasing accuracy can be measured by counting the number of switch errors that occur divided by the number of opportunities for switch errors, known as the “switch error rate.” Switch errors may be classified as long switch errors, point switch errors, or undetermined switch errors. A long switch appears as a large-scale pseudo recombination event in which there are no other local switches surrounding the long switch (e.g., no other switches within three consecutive heterozygous sites). Point switches are small-scale switch errors which appear as two neighboring switch errors (e.g., two switches within three consecutive heterozygous sites, with the pair of switches counted as a point switch). The remaining switches are considered undetermined (e.g., only two sites phased in a small phasing block, so the switch error could not be classified into long or point). Long switches are particularly detrimental to genomic analysis that relies on the phasing of loci since the switch error propagates over larger portions of the genome (e.g., the phasing of a distant locus downstream from a joint switch is unaffected by the joint switch error since the second switch error in the joint switch reverts the nucleotides downstream the joint switch back to their original/proper phasing). Long switch errors, in particular, can manifest themselves as induced and false recombination events in the inferred haplotype compared with the true haplotypes. An important limitation of the use of phase sets has been the presence of long switch errors. These errors directly impact the sensitivity to detect small (e.g., less than about 1 Mb) deletions or amplifications, in particular. In contrast to an isolated phasing error event, switch errors can directly impact the relationship of all downstream loci with respect to an upstream locus and/or all upstream loci with respect to downstream locus. Regions of a genome having low polymorphism or SNV density are particular prone to switch errors when phased.

Switch error rates are generally higher for population-based phasing approaches, which rely on computationally inferring phases from statistical analysis of populations, compared to molecular phasing approaches. Molecular phasing approaches, however, may also be susceptible to switch errors. Many molecular phasing approaches, for instance, may rely on computational construction of synthetic long reads from short reads, which relies on statistically-informed inferences about alignments of the short reads to the genome. For example, haplotyping based on dilution pool sequencing relies on the low molarity of molecules per given partition to reduce the likelihood that one DNA molecule in a partition has overlapping sequence with another. Such assumptions allow at least some haplotypes to be derived, but may introduce switch errors when performing long range phasing (e.g., the phasing of an entire chromosome). Some assumptions about the phase alignment of distant variants may be made in order to find the most likely phase alignment, which could allow the introduction of switch errors.

Phasing approaches which directly rely upon the proximate positioning of two or more loci in an intact chromosome to phase on or more variants at those loci with respect to each other are generally not prone to switch errors since the phase alignment is determined by experimental information that directly ties one variant to another and not on inferences related to the phasing of more distant variants. Thus, even if a phasing error was made using such an approach, the error would not necessarily be propagated to other more distant loci (e.g., downstream loci). Accordingly, such “non-error-propagating” methodologies provide an orthogonal phasing approach to the population-based phasing approaches and molecular phasing approaches which are susceptible to switch errors.

Approaches which are generally non-error-propagating and approaches which are error-propagating are well understood in the art. Examples of non-error-propagating approaches, include but are not limited to chromosome conformation capture (e.g., Hi-C), particularly for proximate (e.g., neighboring) phase sets; single cell-template strand sequencing; and chromosome sequencing (e.g., as obtained by karyotyping or laser capture microdissection). It will be understood that sequencing techniques in which reads can be presumed to come from the same chromosomal homologue, by nature of the experimental setup used to conduct the sequencing (i.e. sequencing approaches that can be experimentally focused on or confined to singular chromosomal homologues), are non-error-propagating approaches. Approaches which are generally susceptible to error propagation (error-propagating) include, but are not limited to, approaches based on sequencing parental sperm and/or polar bodies; dilution pool sequencing; population reference panels; and long read sequencing (e.g., nanopore sequencing), unless the phasing is focused on phase sets within a sufficiently localized region (e.g., within about 50 kb) such that two phase sets can be captured in single reads.

According to some aspects of the invention, non-error-propagating methodologies may be used on targeted regions of DNA to provide accurate phasing of the targeted region. Phasing information derived from non-error-propagating methodologies may be combined with phasing information derived from error-propagating methodologies. For instance, the phasing information derived from a non-error-propagating methodology may be used to identify and correct a switch error in a presumed phasing alignment (e.g., the phasing derived from an error-propagating methodology) and/or to confirm a presumed phasing alignment as the true alignment. The phasing information derived from a non-error-propagating methodology may be used to supply missing phase information in a presumed phasing alignment (e.g., the phasing derived from an error-propagating methodology).

### Ploidy Status

The ploidy status of a chromosome or chromosomal segment may be broadly characterized as euploid (having a normal number of copies) or aneuploid (having an abnormal number of copies). The amount of genetic material present at one or more loci may be used to determine the ploidy status of a genetic sample. Aneuploidies may comprise, for example, unbalanced translocations, uniparental disomy, or other gross chromosomal abnormalities, including copy number variations (CNVs).

### Copy Number Variation

CNVs refer to variations between individual chromosomes in the number of repeats in sections of the genome which generally are repeated. Approximately two-thirds of the entire human genome may be composed of repeats and 4.8-9.5% of the human genome can be classified as CNVs. CNVs are known to at least somewhat predictive of disease phenotypes. CNVs may affect the number of short repeats (e.g., dinucleotide or trinucleotide repeats) or long repeats (e.g., whole gene repeats) and are generally introduced by duplication or deletion events. CNVs are often assigned to one of two main categories, based on the length of the affected sequence. The first category includes copy number polymorphisms (CNPs), which are common in the general population, occurring with an overall frequency of greater than 1%. CNPs are typically small (most are less than 10 kb in length), and they are often enriched for genes that encode proteins important in drug detoxification and immunity. A subset of these CNPs is highly variable with respect to copy number. As a result, different human chromosomes can have a wide range of copy numbers (e.g., 2, 3, 4, 5, etc.) for a particular set of genes. CNPs associated with immune response genes have recently been associated with susceptibility to complex genetic diseases, including psoriasis, Crohn's disease, and glomerulonephritis.

The second class of CNVs includes relatively rare variants that are much longer than CNPs, ranging in size from hundreds of thousands of base pairs to over 1 million base pairs in length. In some cases, these CNVs may have arisen during production of the sperm or egg that gave rise to a particular individual, or they may have been passed down for only a few generations within a family. These large and rare structural variants have been observed disproportionately in subjects with mental retardation, developmental delay, schizophrenia, and autism. Their appearance in such subjects has led to speculation that large and rare CNVs may be more important in neurocognitive diseases than other forms of inherited mutations, including single nucleotide substitutions.

Gene copy number can be altered in cancer cells. For instance, duplication of Chr1p is common in breast cancer, and the EGFR copy number can be higher than normal in non-small cell lung cancer. Cancer is one of the leading causes of death; thus, early diagnosis and treatment of cancer is important, since it can improve the patient's outcome (such as by increasing the probability of remission and the duration of remission). Early diagnosis can also allow the patient to undergo fewer or less drastic treatment alternatives. Many of the current treatments that destroy cancerous cells also affect normal cells, resulting in a variety of possible side-effects, such as nausea, vomiting, low blood cell counts, increased risk of infection, hair loss, and ulcers in mucous membranes. Thus, early detection of cancer is desirable since it can reduce the amount and/or number of treatments (such as chemotherapeutic agents or radiation) needed to eliminate the cancer.

Copy number variation has also been associated with severe mental and physical handicaps, and idiopathic learning disability. Non-invasive prenatal testing (NIPT) using cell-free DNA (cfDNA) can be used to detect abnormalities, such as fetal trisomies 13, 18, and 21, triploidy, and sex chromosome aneuploidies. Subchromosomal microdeletions, which can also result in severe mental and physical handicaps, are more challenging to detect due to their smaller size. Eight of the microdeletion syndromes have an aggregate incidence of more than 1 in 1000, making them nearly as common as fetal autosomal trisomies. In addition, a higher copy number of CCL3L1 has been associated with lower susceptibility to HIV infection, and a low copy number of FCGR3B (the CD16 cell surface immunoglobulin receptor) can increase susceptibility to systemic lupus erythematosus and similar inflammatory autoimmune disorders.

### Determination of Ploidy Status

Various aspects of the invention involve making a determination or call of ploidy status (e.g., calling a CNV) for a subject, cell or population of cells, or other source of genetic material with respect to either a chromosome or chromosomal segment. As used herein, a chromosomal segment may refer to any length or portion of a sequence of a chromosome that can be characterized as having a copy number, including an entire chromosome. A subject may refer to any organism having a genome, preferably a diploid genome. Preferably the subject may be a mammal. According to various aspects the subject is human. Determination of ploidy status may comprise determining the origin of an aneuploidy (i.e. determining which chromosomal homologue comprises the aneuploidy). The origin may be identified, for example, as originating in a maternally inherited or paternally inherited chromosome.

The ploidy status of a chromosome or chromosomal segment may be determined with respect to a reference genetic code. The reference genetic code may correspond to the entire genome of a subject, to an entire chromosome or chromosomes of a subject, or to one or more chromosomal segments (on the same or different chromosomes) of the subject. The reference genetic code may be obtained directly or indirectly from a subject for whom genetic material is being analyzed according to the methods disclosed herein. For example, the reference genetic code may be derived from sequencing normal genetic material (e.g., normal cells or non-cancerous cells) from the subject. Normal genetic material may be genetic material known to be euploid or having previously identified aneuploidies of a known nature. The reference genetic code may be obtained from sequencing somatic cells and/or germline cells of the subject. In some instances, a reference genetic code may be obtained by reconstructing a genetic code from the sequencing of one or more parents or other genetic relatives of the subject from whom the genetic material is being analyzed, particularly if the subject is an embryo or a fetus, according to methods known in the art. See, e.g., WO 2021/067417 to Kumar et al., published on Apr. 8, 2021, which is herein incorporated by reference in its entirety. Constructing the reference genetic code may involve sampling somatic tissue and/or germline tissue of the one or more genetic relatives. Constructing the reference genetic code may involve sampling the subject (e.g., an embryo or fetus) even if only sparse genetic information is obtained. Constructing the reference genetic code may involve sequencing cells obtained from the subject. Constructing the reference genetic code may involve sequencing cell-free DNA (cfDNA), such as through sampling DNA fragments within the subject's blood, within cell culture medium (in the case of an embryo), or within the mother of the subject's blood (in the case of a fetus). In some implementations, the genome of the subject, or at least the genome of the normal cells of the subject, serves as the reference genetic code to which comparisons can be made to determine ploidy status (e.g., of abnormal cells such as tumor cells). In some implementations, the expected genome of a subject (i.e. a genome made up of the specific chromosomes inherited from the subject's parents absent any de novo changes in ploidy status such as a de novo amplification or deletion event) serves as the reference genetic code to which comparisons can be made to determine de novo changes to ploidy status in the subject.

The reference genetic code may not be phased. Preferably, the reference genetic code is entirely phased or at least partially phased. The reference genetic code may be phased by any method known in the art, such as error-propagating phasing approaches. For example, the genetic code may be phased by computational techniques involving reference population panels. The genetic code may be phased by molecular techniques, such as dilution pool sequencing. See, e.g., Choi et al., PLoS Genet. 2018 Apr. 5; 14(4):e1007308 (doi: 10.1371/j ournal.pgen.1007308). The genetic code may be phased by sequencing germline cells of the subject and/or one or more genetic relatives of the subject (e.g., mother and father). See, e.g., WO 2021/067417 to Kumar et al., published on Apr. 8, 2021, which is herein incorporated by reference in its entirety.

Haplotypes are contiguous phased blocks of genomic variants specific to one chromosomal homologue or another. According to various aspects, haplotype blocks may be a priori constructed such that there is certainty or at least a sufficiently high confidence of correct phasing within the haplotype block prior to implementing the methods of the invention described herein. For example, haplotype blocks may be constructed from dilution pool sequencing or long read sequencing in which there is certainty or high confidence that a switch error does not exist within the haplotype block. Obtaining a priori phasing information for a genetic code of interest may comprise obtaining one or more haplotype blocks. In various implementations, one or more of the signals described herein may be averaged across haplotype blocks or across smaller regions or partitions of haplotype blocks.

### Non-Error-Propagating Phasing Approaches

In various implementations, it may be advantageous to combine non-error-propagating phasing approaches with error-propagating phasing approaches. Non-error-propagating phasing techniques can provide an orthogonal source of information to more traditional error-propagating techniques. Error-propagating phasing approaches (e.g., the population-based phasing and molecular phasing approaches described elsewhere herein) may provide a quicker, cheaper, and/or more convenient approach to obtaining large scale sequence and/or phasing information than non-error-propagating approaches. Non-error-propagating approaches may provide more accurate phasing information for targeted regions of a genetic code that allow better determinations of ploidy status (e.g., improve the ability to call CNVs within that targeted region).

The phase alignments that may be obtained from non-error-propagating techniques may be used in a targeted fashion. Depending on the methodology employed, targeted phase correction may focus on particular regions of a genetic code, saving on resources and allowing more efficient implementation of the non-error-propagating methodology or methodologies. For instance, the phasing of specific phase sets relevant to a potential switch error identified from an at least partially phased genome may be used to correct the phasing of those true sets. The phase alignments may be used to re-analyze the entire phasing alignment of a genome, chromosome of interest, or chromosomal segment of interest. The phasing may be used to provide missing phase information for particular variants or chromosomal segments. The phase alignment may be computationally recalculated using the phase alignments in combination with a priori phasing data (e.g., obtained from error-propagating approaches). Methods of incorporating the phasing alignments from the methods described herein with existing phase information are well understood in the art. According to certain aspects of the invention, non-error-propagating techniques may be used in combination with conventional-error propagating techniques to provide an improved process for reconstructing the whole genome, based on the more accurate phasing information obtained. The non-error-propagating techniques may also allow for interpretation of the function of variants within the genome.

Various phasing approaches that would be understood to be non-error-propagating, as described herein, are well known in the art. Described herein are specific, but non-limiting examples, of such techniques that may be used in a non-error-propagating manner.

### Chromosome Conformation Capture (3C)

Chromosome conformation capture (3C) techniques are molecular biology methods used to analyze the spatial organization of chromatin in a cell. 3C methods generally quantify the number of interactions between genomic loci that are nearby in three-dimensional space, including loci which may be separated by many nucleotides in the linear genome sequence (e.g., loci which may be too far apart to capture together via short read and/or long read sequencing). Such interactions may result, for example, from biological functions, such as promoter-enhancer interactions, or from random polymer looping, where undirected physical motion of chromatin causes loci to collide. Interaction frequencies may be analyzed directly, or they may be converted to distances, which may facilitate reconstruct three-dimensional structures. Different 3C-based methods may have different scopes in terms of the genome-wide interactions that may be interrogated. Deep sequencing of material produced by 3C may be used to produce genome-wide interactions maps.

In 3C methods, digestion and subsequent re-ligation of DNA in crosslinked chromatin in cell nuclei allows the detection of spatial proximity between DNA sequences. Certain 3C techniques may be based on high-throughput sequencing technologies. In standard 3C-based protocols, chromatin is usually cross-linked with formaldehyde. The cross-linked chromatin is then fragmented, usually with restriction enzymes, such that the genome is generally cut up approximately every 256 bp or every 4096 bp. In situ ligation then ensures preferential ligations between contacting and crosslinked chromatin fragments. The chromatin is digested such that the crosslinks are reversed resulting in linear and/or circular DNA concatemers carrying shuffled genomic fragments ligated together according to spatial proximity.

3C techniques may comprise classic 3C, 4C, 5C, Hi-C, and ChIA-PET methodologies. Classic 3C, often referred to as a “one-to-one” approach, uses PCR to amplify and quantify specifically targeted ligation junctions. 4C, often referred to as a “one-to-all” approach, is similar to the classic 3C technique, except that a second round of digestion and ligation is performed to result in small DNA circles. Primers designed to a specific anchor sequence can then be used in inverse PCR to amplify all contacting sequences that formed ligation products with the anchor sequence, although modern methods may avoid the need for amplification. The contacting sequences can then be sequenced by any suitable means. 5C, often referred to as a “many-to-many” approach, hybridizes and then ligates primers complementary to fragments of interest to the 3C ligation products to create carbon-copies of the junctions of interest, to the extent present. Universal PCR primaries complementary to the original primers' tails are then used to amplify the ligation products of interest which may be sequenced by any suitable means. Hi-C, often referred to as an “all-to-all” approach, uses restriction enzymes that leave overhangs that are filled with biotin-labeled nucleotides. After blunt-end ligation, the ligation products are sheared to reduce fragment size and streptavidin is used to pull-down the biotin-containing fragments to create an enriched library which is then sequenced, usually by NGS technology. Hi-C renders a matrix of pair-wise interactions frequencies between fragments across the entire genome. The resolution can be improved by using higher restriction site density and/or by increasing sequencing depth, with the sequencing of x² more pairs generally resulting in an x-fold improvement in resolution. With Hi-C, in particular, measurements corresponding to individual variants of interest may be sparse, but because measurements throughout the chromosome are largely consistent, when used in aggregate they can improve the phasing across the chromosome. ChIA-PET is a combination of Hi-C with chromatin immunoprecipitation (ChIP). A specific antibody is used to pull down ligation junctions bound by a chromatin protein of interest prior to biotinylating and ligating the fragment ends. Other chromosome conformation capture techniques that are known in the art include tethered conformation capture (TCC), DNase Hi-C or Micro-C, targeted chromatin capture (T2C), capture Hi-C(Chi-C), HiCap, and Capture-C. Various methods for performing chromosome conformation capture may be performed, as described, for example, in Denker, et al., Genes Dev. 2016 Jun. 15; 30(12):1357-82 (doi: 10.1101/gad.281964.116); de Wit, et al., Genes Dev. 2012 Jan. 1; 26(1):11-24 (doi: 10.1101/gad.179804.111); McCord et al., Mol Cell. 2020 February 20; 77(4): 688-708) (doi:10.1016/j.molcel.2019.12.021); or Belton et al., Methods. 2012 November; 58(3):268-76 (doi: 10.1016/j.ymeth.2012.05.001), each of which is herein incorporated by reference in its entirety.

Chromosome conformation capture techniques can be used to phase genomes in a non-error-propagating manner. Because there is a much larger probability for loci on the same chromosomal homologue to be ligated together, based on their inherent spatial proximity, than for loci on two homologous chromosomes to be ligated together, it may be assumed that the overall distribution of ligation fragments generated by 3C technologies will comprise a predominance of variants from the same chromosomal homologue relative to variants from the two or more different homologues. Furthermore, the effect becomes more predominant the closer to each other the variants or phases sets are. Thus, chromosome conformation capture techniques, such as Hi-C, can be used to align two phases, particularly two neighboring phase sets, without the concern of introducing a switch error.

The distribution of fragments (ligation products) obtained from a chromosome conformation capture methodology may be analyzed to determine whether the distribution supports two phase sets being in phase or out of phase. The fragments may be filtered to select those fragments which comprise at least one variant from each phase set. The fragments may be grouped into subgroups corresponding to different sets of variants that support the same haplotype call, although each fragment may not comprise the same variants. In some implementations, fragments may be filtered for only those fragments that comprise each variant from one or both phase sets. Each phase set may be assigned a presumptive phase or haplotype such that there is a presumptive phase alignment. If no a priori phase determination has been made then a phase alignment may be randomly assigned. The selected fragments and/or subgroups may be characterized as concordant or discordant with respect to the presumptive phase alignment. For example, if all of the variants detected within a fragment are from the same presumptive haplotype then the fragment may be considered concordant with the presumptive phase alignment and otherwise the fragment may be considered discordant. Given the substantially higher probability of the fragments including variants from the same haplotype or chromosomal homologue, particularly for proximate variants, the distribution of fragments/subgroups may be expected to be either heavily skewed toward a predominance of concordant or discordant fragments. A predominance of concordant fragments/subgroups suggests the presumptive phase alignment is correct, whereas a predominance of discordant fragments suggests the presumptive phase alignment is incorrect. The amount of skew can be quantified by calculating a probability of observing the skew by chance. For example, a binomial probability may be calculated for the probability of observing the measured distribution by chance, wherein each measurement has a fixed probability of being concordant or discordant. The fixed probability may be set as a floor as 50% suggesting the ligation of the phase sets is entirely random. Alternatively, the fixed probability of phase sets from the same haplotype being in the same fragment may be set higher (e.g. 60%, 70%, 75%, 80%, 90%, 95%, 99%, 99.9%, etc.) to account for the higher probability expected from the spatial proximity. Higher fixed probabilities may be more useful for smaller numbers of measurements, whereas lower fixed probabilities may suffice for larger numbers of measurements. If there is a high confidence that the observed distribution is not merely the result of chance (e.g., the measurements are statistically significant with respect to a 95% confidence interval) then the phase sets may accurately aligned based on the chromosome conformation data.

### Single-Cell Template Strand Sequencing

Single-cell template strand sequencing (Strand-seq) is a single-cell sequencing technology that resolves the individual homologs within a cell by restricting sequence analysis to the DNA template strands used during DNA replication. The method relies on the directionality of DNA (distinguished by its 5′-3′ orientation) by culturing cells in a thymidine analog during a single round of cell division to label nascent DNA strands, which can then be selectively removed from analysis. Each single-cell library is multiplexed for pooling and sequencing, and the resulting sequence data are aligned, mapping to either the minus or plus strand of the reference genome, to assign template strand states for each chromosome in the cell. See, e.g., Porubsky et al., Genome Res. 2016 November; 26(11):1565-1574 (doi: 10.1101/gr.209841.116); Sanders et al., Nat Protoc. 2017 June; 12(6):1151-1176 (doi: 10.1038/nprot.2017.029), each of which is herein incorporated by reference in its entirety. Because sequencing can be confined to a single strand, the technique may be used as a non-error-propagating method described herein.

### Chromosomal Isolation

Any technique which physically isolates one chromosomal homologue from another prior to sequencing may be considered a non-error-propagating approach to phasing, since the sequence reads may all be presumed to be derived from the same homologue. Sequencing of chromosomes obtained, for example, via karyotype or laser capture microdissection may be used for the non-error-propagating techniques described herein. See, e.g., Kang et al., Cytogenet Genome Res. 2017; 152(4):204-212 (doi: 10.1159/000481790), which is herein incorporated by reference in its entirety.

### Sequencing Methodologies

Various methods of DNA sequencing are well known in the art and may be used to implement the methods described herein unless dictated otherwise by context. DNA sequencing may comprise for example Sanger sequencing (chain-termination sequencing). DNA sequencing may comprise use of next-generation sequencing (NGS) or second generation sequencing technology, which is typically characterized by being highly scalable, allowing an entire genome to be sequenced at once. NGS technology generally allows multiple fragments to be sequenced at once allowing for “massively parallel” sequencing in an automated process. DNA sequencing may comprise third generation sequencing technology (e.g., nanopore sequencing or SMRT sequencing), which generally allows for obtaining longer reads than obtainable via second generation sequencing technology. Sequencing may comprise paired-end sequencing, where feasible, in which both ends of a DNA fragment are sequenced, which may improve the ability to align the reads to a longer sequencing. DNA sequencing may comprise sequencing by synthesis/ligation (e.g., ILLUMINA® sequencing), single-molecule real time (SMRT) sequencing (e.g., PACBIO® sequencing), nanopore sequencing (e.g., OXFORD NANOPORE® sequencing), ion semiconductor sequencing (Ion Torrent sequencing), combinatorial probe anchor synthesis sequencing, pyrosequencing, etc.

Shotgun sequencing refers to a method of sequencing random DNA strands from a genome or large genetic sample. DNA is broken up randomly into numerous small segments, which are sequenced (e.g., using the chain termination method) to obtain reads. Multiple overlapping reads for the target DNA are obtained by performing several rounds of this fragmentation and sequencing. Computational algorithms then use the overlapping ends of different reads to assemble the reads of the random segments into a continuous sequence. Shotgun sequencing may be used for whole genome sequencing. Any suitable form of sequencing, including those describe herein, may be used to identify variants (e.g., SNPs) in a subject which may subsequently be used as the basis for measuring genetic signals indicative of ploidy status for a chromosomal segment comprising that variant, as described elsewhere herein. According to certain aspects of the invention, hierarchical sequencing may be used for whole genome sequencing.

### Data Collection

Genetic material for analysis by the methods described herein may be obtained from various sources, including somatic cells (e.g., white blood cells, cells from tissue biopsies), germ cells (e.g., sperm, eggs, polar bodies), and cell-free DNA. Genetic material may be collected directly from the subject for whom the genome is being analyzed and/or from genetic relatives of the subject (e.g., the mother and/or father). According to various implementations, a genetic signal indicative of ploidy status, such as an allele balance signal or depth of read signal, may be obtained from cell-free DNA (cfDNA) derived directly from the subject. Cell-free DNA is DNA that is found outside a cell, e.g., freely circulating in the bloodstream or in the cell culture medium of cultured cells, such as embryos grown for in vitro fertilization (IVF).

Various implementations of the methods described herein may involve obtaining and/or sequencing cell-free DNA. Cell-free DNA may comprise cell-free fetal DNA (cffDNA). Cell-free DNA may comprise circulating tumor DNA (ctDNA). Cell-free DNA may provide a relatively abundant source of genetic material that can be obtained from a non-invasive or minimally invasive procedure, such as sampling cell culture medium or drawing blood from a subject. Cell-free DNA may provide ample genetic information for whole genome sequencing of the subject from whom the cell-free DNA is derived. See, e.g., Kitzman et al., Sci Transl Med. 2012 Jun. 6; 4(137):137ra76 (doi: 10.1126/scitranslmed.3004323). For instance, shotgun sequencing of cell-free DNA may be used to sequence one or more chromosomes of the subject. The genetic material from the subject may have cells of a consistent genetic profile or having cells with different genetic profiles (e.g., normal cells and tumor cells). In some instances, the genome of the subject may be reconstructed based on sequencing of genetic material obtained directly from the subject and sequencing of one or more genetic relatives. See, e.g., WO 2021/067417 to Kumar et al., published on Apr. 8, 2021, which is herein incorporated by reference in its entirety.

Cell-free fetal DNA (cffDNA) is fetal DNA that circulates freely in the maternal blood. Thus, cffDNA may be obtained from maternal blood sampled, for example, by venipuncture. Analysis of cffDNA is a method of non-invasive prenatal diagnosis that may be ordered for pregnant women. cffDNA originates from placental trophoblasts. Fetal DNA is fragmented when placental microparticles are shed into the maternal blood circulation. Because, cffDNA fragments, which are approximately 200 bp in length, are significantly smaller than maternal DNA fragments, they can be distinguished from maternal DNA fragments. Approximately 11-13.4% of the cell-free DNA in maternal blood is cffDNA, although the amount varies widely between pregnant women. cffDNA generally becomes detectable after five to seven weeks gestation and the amount increases as the pregnancy progresses. The quantity of cffDNA in maternal blood diminishes rapidly after childbirth, generally being no longer detectable about 2 hours after delivery. Analysis of cffDNA may provide earlier diagnosis of fetal conditions than other techniques. cffDNA may be analyzed, for example, by massively parallel shotgun sequencing (MPSS), targeted massive parallel sequencing (t-MPS), and SNP assays.

ctDNA is tumor-derived fragmented DNA in the bloodstream that is not associated with cells. Because ctDNA may reflect the entire tumor genome, it has gained traction for its potential clinical utility. “Liquid biopsies” in the form of blood draws may be taken at various time points to monitor tumor progression throughout a treatment regimen. ctDNA originates directly from the tumor or from circulating tumor cells (CTCs), which are viable, intact tumor cells that shed from primary tumors and enter the bloodstream or lymphatic system. The precise mechanism of ctDNA release remains unclear. The biological processes postulated to be involved in ctDNA release include apoptosis and necrosis from dying cells, or active release from viable tumor cells. Studies in both human (healthy and cancer patients) and xenografted mice show that the size of fragmented cfDNA is predominantly 166 bp long, which corresponds to the length of DNA wrapped around a nucleosome plus a linker. Fragmentation of this length might be indicative of apoptotic DNA fragmentation, suggesting that apoptosis may be the primary method of ctDNA release. The fragmentation of cfDNA is altered in the plasma of cancer patients. In healthy tissue, infiltrating phagocytes are responsible for clearance of apoptotic or necrotic cellular debris, which includes cfDNA. cfDNA in healthy patients is only present at low levels, but higher levels of ctDNA in cancer patients can be detected with increasing tumor sizes. This possibly occurs due to inefficient immune cell infiltration to tumor sites, which reduces effective clearance of ctDNA from the bloodstream. Comparison of mutations in ctDNA and DNA extracted from primary tumors of the same patients has revealed the presence of identical cancer-relevant genetic changes, allowing for the possibility of analyzing ctDNA in order to analyze the genetic makeup of tumor cells. Accordingly, ctDNA may be used for earlier cancer detection and treatment follow up monitoring.

According to various aspects of the invention, the non-error-propagating phasing techniques described elsewhere herein are performed on cellular DNA (not cell-free DNA) such that intact chromosomes are isolated or effectively isolated to provide accurate phasing (e.g., correct for any switch errors). In some implementations, single cell sequencing may be performed on one or more cells to obtain the data described herein. The genetic data obtained using the non-error-propagating phasing techniques may or may not be sufficient to independently construct the subject's genome or independently provide a sufficient reference genome. The genetic data obtained from conventional sequencing techniques (e.g., whole genome shotgun sequencing, such as on cell-free DNA) in combination with error-propagating phasing approaches may be advantageous in providing a depth and/or range of genetic information. The genetic data obtained from the non-error-propagating phasing approaches (which may be performed on cellular DNA) may be advantageous in providing more accurate phasing of various phase sets, particularly proximate or neighboring phase sets. Accordingly, the use of these orthogonal sources of information together can be advantageous.

According to some aspects of the invention, the sequencing of cellular DNA may be performed on blood cells (e.g., white blood cells) or other cells collected through noninvasive or minimally invasive techniques (e.g., cells found in saliva). Thus, the sequencing of cell-free DNA and cellular DNA may be performed entirely by non-invasive or minimally invasive procedures, such as through blood collection. The cell-free DNA and cellular DNA may be isolated from the same or different samples (e.g., body fluid sample such as a blood sample or saliva sample). For example, the cell-free DNA may comprise ctDNA and the cellular DNA may comprise white blood cell DNA (which should provide normal genetic material except in cases of leukemia).

According to some aspects of the invention, sequencing cellular DNA may involve isolating one or more cells from a fetus or embryo according to methods which are well understood in the art. Such approaches typically require invasive techniques that may impose a risk to the embryo or fetus. According to preferred aspects of the invention, cellular DNA used for non-error-propagating phasing approaches may be obtained using non-invasive or minimally invasive techniques, such as blood draws or sperm collection. Although non-invasive or minimally invasive techniques for sequencing cellular DNA may not be possible on the subject's own cells in the case of an embryo or fetus, sequencing cellular DNA may be performed on a genetic relative of the fetus (e.g., a mother and/or a father). Because the non-error-propagating phasing may be used only to provide accurate phasing of phase sets and not necessarily to independently construct the reference genetic code and/or generate signals indicative of ploidy status, the true phasing of the subject's genome may be deduced from the true phasing of the genomes of one or more genetic relatives, who have inherited at least some of the same haplotypes as the subject. Accordingly, the methods described herein may be conducted on genetic material obtained through entirely non-invasive or minimally invasive methods, including when the subject is an embryo or a fetus.

### Genetic Signals Indicative of Ploidy Status

As used herein, a “signal” may refer to one or more measurements that may provide information on the genetic composition of an interrogated genetic sample. The measurements may be raw measurements or processed measurements derived, for example, from mathematical analysis of one or more raw measurements. The signal may be obtained from sequencing data. The signal may be, for example, an allele balance signal or depth of read signal, as described elsewhere herein. The signal may correspond to a value along a continuous or discrete number spectrum. A signal may be indicative of genetic information at one specific locus. A signal may be averaged from the signals measured across a plurality of loci.

A genetic locus is a specific, fixed position on a chromosome. Loci identify the chromosomal positions of particular genes and genetic markers. As used herein a locus of interest may refer to a locus within the genetic material being analyzed for which one or more measurements may be mapped to the locus in order to derive a signal indicative of the genetic composition of the genetic material. A variant of interest may refer to a locus of interest in which there is a difference in the genetic composition at the locus of interest between two or more chromosomal homologues within the genetic material. A SNP may be a variant of interest. As used herein, a “phase set” may refer to a set of one or more neighboring variants of interest for which a phase alignment with another phase set may be determined according to the methods described herein. In some instances, a phase set may correspond to a haplotype block or a chromosomal region larger than a haplotype block (e.g., two or more neighboring haplotype blocks). For example, the phase set may comprise 2, 5, 10, 50, 100, 500, 1,000, 5,000, or more variants. In some instances, a phase set may consist of a single variant. The two phase sets being aligned may or may not have the same number of variants of interest. Determining the phase alignment of one phase set with another phase set may comprise determining that the two phase sets are in phase (i.e. the variants of interest within each phase set belong to the same chromosomal homologue) or that the two phase sets are out of phase (i.e. that the variants of interest within a first phase set do not belong to the same chromosomal homologue as the variants of interest within the second phase set).

According to some specific aspects, the phase sets may be neighboring phase sets. For instance, the first phase set may have a variant of interest that is no further than approximately 1,000, 5,000, 10,000, 50,000, 100,000, 500,0000, 1,000,000, 5,000,000, 10,000,000, 50,000,000, 100,000,000, or 250,000,000 bp from a variant of interest in the neighboring phase set. The neighboring phase sets may be defined to encompass the variants of interest on either side of a potential switch error. A potential switch error may be identified as possibly occurring between two haplotype blocks. According to some specific aspects, a site where one or more signals suggests a shift between chromosomal segments from a euploid segment to an aneuploid segment or vice-versa may be identified as a potential switch error. According some specific aspects, a site where one or more signals suggests a change in copy number relative to a neighboring segment may be identified as a potential shift error. According some specific aspects, a site where one or more signals suggests a shift between chromosomal segments of different aneuploid status (e.g., from trisomy to monosomy or vice-versa) may be identified as a potential switch error.

Allele balance (synonymous with allelic balance, allele fraction, or allelic fraction) refers to the proportion of reads from a set of sequencing data that cover a variant's location that support the variant. For example, if 100 reads are mapped to the locus of a particular variant, of which 25 support the variant and 75 do not, then the variant would have an allele balance of 0.25. Heterozygous loci may be filtered for a minimum depth of read for inclusion in allele balance data. The relative proportion of one variant relative to another may indicate a difference in copy number of the locus between different chromosomal homologues in the genetic sample. Comparing the copy number expected based on the reference genetic code to the number detected may indicate, for example, whether an amplification or deletion event has occurred on one of the chromosomal homologues (e.g., in all or at least a portion of the cells from which the genetic sample was derived). An allele balance signal measured over a plurality of variants can provide a signal for the balance of a haplotype or chromosome, based on the assignment of the alleles to a haplotype or chromosomal homologue. Because allele balance thereby becomes dependent on the phasing of the variants (i.e. whether a relatively high or low proportion of an allele supports a high or low proportion of a chromosomal homologue depends on its phasing), the allele balance signal may be altered by a phasing error, such as a switch error. Thus, phase correction may directly translate to an allele balance correction such that a true allele balance signal is obtained from correcting the phase alignment. As used herein, “correcting” a phase alignment or allele balance signal may be used to refer to comparing a phase determination to an a priori or otherwise presumed phase determination, regardless of whether an incorrect phase is actually identified and changed, or to supplying missing phase information, unless dictated otherwise by context (e.g., “correcting an error”).

Depth of read refers to the number of sequencing reads that map to a given locus over the course of one or more sequencing runs. The depth of read signal (or depth signal) may be normalized over a total number of reads. Depth of read can be expressed in variety of different ways, including but not limited to an absolute number of reads mapped by a sequencer to the particular locus or the percentage or proportion of reads mapped to that locus. Thus, for example in a highly parallel DNA sequencer such as an ILLUMINA HISEQ®, which, e.g., produces a sequence of 1 million clones, the sequencing of one locus 3,000 times results in a depth of read of 3,000 reads at that locus. The proportion of reads at that locus is 3,000 divided by 1 million total reads, or 0.3% of the total reads. In general, the greater the depth of read at a locus, the closer the allele balance signal at the locus tend to be to the true allele balance in the original genetic sample. Loci may be filtered for a minimum depth of read for inclusion in depth of read data. The depth of read of a particular variant, particularly when normalized against a total number of reads, may indicate the relative number of copies of that variant compared to other variants. Comparing the relative number of copies for a variant to one or more benchmarks for known numbers of copies, such as from a reference genetic code, may indicate, for example, whether an amplification or deletion event has occurred on one of the chromosomal homologues (e.g., in all or at least a portion of the cells from which the genetic sample was derived).

Noise may be introduced into the signals by a number of mechanisms, including, for example, by stochastic events due to sampling, GC bias, and/or the uneven distribution of variants across the genome, in addition to any copy number abnormality. The signals described herein may generally be averaged across a plurality of neighboring loci. For example, the plurality of neighboring loci may comprise 2, 3, 4, 5, 10, 15, 20, 25, 30, 40, 50, 100, 500, 1,000, 5,000, or more loci. The selection of loci may depend on their density with the region of interest. For example, the plurality of neighboring loci may comprise all loci within a region of at least about 100,000, 200,000, 300,000, 400,000, 500,000, 750,000, 1,000,000, 50,000,000, or 100,000,000 bp. The plurality of neighboring loci may comprise all loci within a region no greater than about 50,000, 100,000, 200,000, 300,000, 400,000, 500,000, 750,000, 1,000,000, 50,000,000, or 100,000,000 bp. The range of the neighboring loci may be selected such that the loci are presumed to reside on the same chromosome. Accordingly, the true signal for allele balance or depth of read for each of the loci should be the same unless an aneuploidy exists with respect to only some of the loci within the selection. Averaging across neighboring loci may, therefore, reduce the noise in the signals described herein.

### Combining Allele Balance and Depth of Read

According to various aspects of the invention, an allele balance signal and a depth of read signal may be used in combination to make a determination of ploidy status. Allele balance and depth of read may each be individually indicative of a ploidy status determination, as described elsewhere herein. However, as the noise from these signals are at least somewhat independent, the noise in allele balance relating to variations in the sequenced number of specific DNA molecules that overlap an interrogated site and the noise in depth of read relating to variations in the sequenced total number of DNA molecules that overlap an interrogated site, the signals can provide orthogonal sources of information to one another, improving the signal-to-noise ratio and allowing more accurate ploidy status determinations. The combination may be particularly useful in scenarios in where there are an intermediate number of reads (i.e. enough reads that an allele balance at a locus can be finely enough determined, but not so many reads that a depth of read signal becomes dispositive). The allele balance signal may be corrected via a non-error-propagating phasing approach to provide a true allele balance signal according to the methods described elsewhere herein.

The signals may be used in combination according to various ways, as are understood in the art. For example, the signals may be used in combination together by way of multivariate logistical regression, loglinear modeling, neural network analysis, n-of-m analysis (aneuploidy indicated if at least “n” number of criteria out of a total of “m” number of criteria are satisfied), decision tree analysis, random forests analysis, rule sets, Bayesian methods, neural network methods, multiplication, addition, etc. Some methods of using the signals together may comprise combining the two signals into a single composite signal through mathematical operations. For example, the signals may be multiplied together or added together. In various implementations, one or both of the signals may be multiplied by a scalar. For instance, the signals may be normalized relative to one or more measures of noise, such as the standard deviation or variance measured in the signal (e.g., across multiple chromosomal positions where the signal is measured and/or across multiple runs of the analysis).

For each signal and/or combination of signals, one or more thresholds levels or values of the signal may be selected as cutoffs to distinguish different copy numbers of a locus or chromosomal segment. For example, a threshold may be selected to distinguish a locus that exists in trisomy (three copies of the locus) vs. a locus that exists in disomy (two copies of the locus) and/or a threshold may be selected to distinguish a locus that exists in monosomy (one copy of the locus) vs. a locus that exists in disomy. The signal may be offset or otherwise normalized against the signal (e.g., mean signal value) for a different copy number, such as the euploid copy number. For instance, the signal may be configured such that a level of 0 indicates a euploid ploidy status and sufficient deviations therefrom are indicative an aneuploid ploidy status. Different thresholds may be selected to be indicative of different copy numbers.

The use of the signals individual and/or in combination may be characterized by the probability that a signal is able to correctly distinguish between two populations having different copy numbers, such as a euploid population and an aneuploid population. The probability may be characterized, for example, as the probability that using a threshold value of the signal correctly identifies which population a variant should be assigned to. The probability may characterized by the probability of a true positive, a false positive, a true negative, and/or a false negative. A probability based on an individual signal is the individual probability. A probability based on using the two signals in combination is a joint probability. For example, the probability of a true positive aneuploid call is the probability that an aneuploid is accurately identified as an aneuploid based on the criteria for a positive call using the two signals in combination. The use of the allele balance signal and depth of read signal in combination may generally provide higher joint probabilities of true positives and/or true negatives relative to the individual probabilities and/or provide lower joint probabilities of false positives and/or false negatives relative to the individual probabilities, as demonstrated elsewhere herein.

The ability of a threshold to sufficiently distinguish two populations (e.g., euploidy vs aneuploidy) can be established using Receiver Operating Characteristic (ROC) analysis as is known in the art. The area under an ROC curve can provide a measure of the quality of the using the signal to distinguish the two populations, irrespective of the particular threshold. To draw an ROC curve, the true positive rate (TPR) and false positive rate (FPR) are determined as the decision threshold is varied continuously. A perfect test for distinguishing the two populations will have an area under the ROC curve of 1.0; a random test will have an area of 0.5. Preferably, the signal(s) provide an ROC curve area greater than 0.5, preferably at least 0.6, more preferably 0.7, still more preferably 0.75, even more preferably at least 0.8, still even more preferably at least 0.9, and most preferably at least 0.95.

Specific thresholds may be selected to provide an acceptable level of sensitivity (true positive rate) and specificity (true negative rate). For example, a threshold may be selected so that the false positive rate is approximately equal to the false negative rate. Such thresholds may be assumed to be for example, one half of the average signal level for an aneuploidy (or particular aneuploidy status) when offset against the average signal level for the euploidy (or not the aneuploidy status). According to certain aspects, a threshold may be selected to provide a specificity greater than 0.5, preferably at least 0.6, more preferably at least 0.7, still more preferably at least 0.8, even more preferably at least 0.9 and most preferably at least 0.95. According to certain aspects, a threshold may be selected to provide a sensitivity greater than 0.5, preferably at least 0.6, more preferably at least 0.7, still more preferably at least 0.8, even more preferably at least 0.9 and most preferably at least 0.95. According to certain aspects, a threshold may be selected to provide an odds ratio different from 1, preferably at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less.

Specific thresholds may be selected independently from measurements of one of the two populations for which a threshold is distinguishing. For example, the threshold for distinguishing an aneuploid variant from a euploid variant may be set as a particular percentile of the euploid population, e.g., the 60^{th}, 70^{th}, 80^{th}, 90^{th}, 95^{th}, 99^{th}, etc. percentile (assuming the aneuploid signal should be greater than the euploid signal), which may be established based on an acceptable level of false positives. Alternatively, the threshold may be set as a particular percentile of the aneuploid population, e.g., the 1^{st}, 5^{th}, 10^{th}, 20^{th}, 30^{th}, 40^{th}, etc. percentile (assuming the aneuploid signal should be greater than the euploid signal), which may be established based on a acceptable level of false negatives. In some instances, the euploid signal may be used to establish a threshold if there is more data available for characterizing the euploid population.

The populations described herein may be any population of measurements. Preferably, the populations may be populations of measurements obtained from the same sequencing experiment on the same genetic material. Defining the populations as such may minimize noise within the populations. Such populations may include measurements over different loci sharing the same ploidy status. Populations, however, may be defined to refer to or include measurements from different sequencing experiments on the same sample of genetic material, different sequencing experiments on a different sample of the same genetic material, and/or different sequencing experiments on different genetic material (e.g., different genomes).

In various implementations, a baseline signal may be established from the same sequencing data for which a potential aneuploid is to be identified. For instance, the baseline signal (e.g., a mean signal value) may be established based on signal measurements for one or more chromosomal segments that are known or confirmed to be euploid. Signals for other segments of the chromosome that are being interrogated for a identification of a potential aneuploid may be offset by this baseline signal as is described elsewhere herein. Doing so may allow easier comparison of the different signal types.

According to some aspects, a population may be assumed to possess a normal distribution. Accordingly, characteristics of the population may be computationally established from a mean signal value for the population, and optionally a measure of noise or variance/standard deviation within the population. Two populations (e.g., a euploid population and an aneuploid population) may be presumed to have approximately the same variance/standard deviation, which may simplify the theoretical characterization of the populations, as described elsewhere herein. Particularly where two populations are determined from the same sequencing experiment (e.g., on different segments of a chromosome) the noise within each signal may be assumed to be substantially the same.

According to some implementations, the allele balance signal and the depth of read signal may be obtained from the same sequencing experiment. In other words, reads from a single experiment may be mapped to variants within a reference genetic code and the relative number of reads mapped to different alleles for the same variant may be used to obtain an allele balance signal, while the total number of reads mapped to a specific variant (optionally, normalized against a total number of reads from the experiment) may be used to obtain a depth of read signal. In various applications, both signals will be obtained from sequencing cell-free DNA, as described elsewhere herein. According to other implementations, the allele balance signal and depth of read signal may be obtained from different sequencing experiments. The different sequencing experiments may be conducted on the same sample of genetic material or different samples of genetic material. When different samples are used, the genetic material may be obtained from the same source (e.g., cell-free DNA) or from different sources (e.g., cell-free DNA vs. cellular DNA or different cell types). In situations where the allele balance signal and/or depth of read signal is obtained from cellular DNA, the source of the genetic material (the particular sample and/or cell type) may be the same as that used for any non-error-propagating phasing, as described elsewhere herein, or may be different.

### Applications

Various potential applications of making a ploidy status determination for a sample of genetic material (e.g., for a genome) are possible. Described herein are several specific, but non-limiting, examples of how such determinations can be used to drive subsequent decisions and/or further analysis or treatments.

### Genetically Profiling Tumors Having Chromosomal Instability

Genomic instability of tumor cells is often associated with poor patient outcome and resistance to targeted cancer therapies. The accumulation of genetic and epigenetic lesions in response to environmental exposures to carcinogens and/or random cellular events often results in the inactivation of tumor suppressor genes that play critical roles in the maintenance of cell cycle, DNA replication and DNA repair. Loss or inhibition of cellular DNA repair mechanisms often results in an increased mutation burden and genomic instability. CNVs are prevalent across many types of cancer types and may cause the gain of oncogenes and/or loss of tumor suppressors associated with disease progression and therapeutic response or resistance. Genomic instability is associated with sub-clonal heterogeneity and is frequently observed in solid tumors between different lesions, within the same tumor, and even within the same solid biopsy site. Such tumor cell heterogeneity can complicate therapeutic intervention designed around single molecular targets. Genome-wide CNV profiles can be used to characterize genomic instability, However, assessment of genomic instability in bulk tumor or biopsy can be complicated due to sample availability as well as noise stemming from surrounding tissue contamination or tumor heterogeneity. Tumors associated with increased genomic instability have been shown to respond to specific types of therapies, including, for example, platinum-based chemotherapy and PARP inhibitors. See, e.g., Greene et al., PLoS One. 2016 Nov. 16; 11(11):e0165089 (doi: 10.1371/j ournal.pone.0165089), which is herein incorporated by reference in its entirety.

Poly ADP ribose polymerases (PARPs), nuclear enzymes found in almost all eukaryotic cells, catalyze the transfer of ADP-ribose units from nicotinamide adenine dinucleotide (NAD+) to nuclear acceptor proteins, and are responsible for the formation of protein-bound linear and branched homo-ADP-ribose polymers. Activation of PARP and resultant formation of poly(ADP-ribose) can be induced by DNA strand breaks after exposure to chemotherapy, ionizing radiation, oxygen free radicals, or nitric oxide (NO). Several forms of cancer are more dependent on PARP than regular cells, making PARP an attractive target for cancer therapy, independent of the specific cancer indication. Also, because PARP is associated with the repair of DNA strand breakage in response to DNA damage caused by radiotherapy or chemotherapy, it can contribute to the resistance that often develops to various types of cancer therapies. Consequently, inhibition of PARP may retard intracellular DNA repair and enhance the antitumor effects of cancer therapy. Indeed, in vitro and in vivo data show that many PARP inhibitors potentiate the effects of ionizing radiation or cytotoxic drugs such as DNA methylating agents. The PARP family of enzymes is extensive and competitive inhibitors of PARP are known. Approved PARP inhibitors include olaparib (Lynparza®, AstraZeneca); rucaparib (Rubraca®, Clovis Oncology); niraparib (Zejula®, Tesaro); and talazoparib (Talzenna®, Pfizer). Other PARP inhibitors being studied include veliparib (ABT-888, AbbVie); pamiparib (BGB-290) (BeiGene, Inc.); CEP 9722 (Cephalon); E7016 (Eisai); and 3-aminobenzamide.

Platinum-based chemotherapeutic (antineoplastic drugs, informally called “platins”) are coordination complexes of platinum, including cisplatin, oxaliplatin, and carboplatin, as well as several proposed drugs under development. Platinum-based chemotherapeutics cause crosslinking of DNA as monoadduct, interstrand crosslinks, intrastrand crosslinks or DNA protein crosslinks that inhibits DNA repair and/or DNA synthesis.

Other forms of treatment that are appropriate for cancers exhibiting chromosomal instability are understood in the art. Accordingly, the methods described herein may relate to identifying genetic signatures in subjects having cancer that are indicative of chromosomal instability and, therefore, suitable for classes of therapeutics targeting genetic mechanisms (e.g., inhibiting the repair of DNA so that the damaged DNA may be more effectively targeted). These therapeutics may be agnostic to the specific type of cancer. Accordingly, the methods described herein may be performed on subjects diagnosed as having or suspected of having cancer prior to or concurrently with specific cancer diagnoses and/or tissue biopsies. Advantageously, the methods described herein may be performed based on genetic material collected entirely from noninvasive or minimally invasive procedures, such as blood draws. The genetic analysis described herein may be performed concurrently with other routine analyses and/or cancer diagnoses or assessment based on the same or different biological samples collected at the same time.

According to specific aspects of the invention, an allele balance signal and/or depth of read signal (e.g., used in combination) may be obtained from a sample of genetic material collected from the subject. The signals may be obtained from cell-free DNA which comprises or is suspected of comprising ctDNA. The signal may be obtained from cellular DNA, such as tumor tissue. If an allele balance signal is used, the true signal may be determined by correcting the allele balance signal using a non-error-propagating phasing technique, as described elsewhere herein. The non-error-propagating phasing technique may be performed on cellular DNA. The cellular DNA may be obtained from blood cells (e.g., white blood cells). According to some aspects in which the one or more signals indicative of ploidy status are obtained from cellular DNA and non-error-propagating phasing is performed on cellular DNA, the same source of cellular DNA may be used for both. In some implementations, cell-free DNA for obtaining the genetic signals of ploidy status and cellular DNA for performing the non-error-propagating phasing are obtained from the same biological sample (e.g., blood draw). A ploidy status determination may be made from the one or more signals to evaluate the ploidy status of the assessed DNA (e.g., the cell-free DNA). The determination may be made with respect to a reference genetic code (e.g., a normal cell genetic code), as described elsewhere herein. The ploidy status may be determined for one or more chromosomal segments. The detection of one or more chromosomal segments exhibiting CNVs may be used to identify one or more regions of the genome displaying chromosomal instability. The identification of such regions may be used to indicate the presence of tumors that are susceptible to treatment with therapeutics that exploit chromosomal instability, such as treatment with PARP inhibitors and/or platinum-based chemotherapeutics. According to some aspects, the ploidy status determination is used to treat the subject (e.g., by administering the treatment in vivo). According to some aspects of the invention, the ploidy status determination is used to treat one or more cells in vitro. The one or more cells may comprise cancer cells. The cells may have been cultured from a subject having or suspected of having cancer (e.g., grown from a tumor biopsy). The cells may comprise cells from an cancer cell line (e.g., artificially induced to replicate a cancer). The cells may comprise a mixture of normal cells and cancerous cells.

### De Novo or Inherited CNV Detection

The methods described herein may be used to detect variations in ploidy status (e.g., CNVs) in a subject. According to some aspects of the invention, an allele balance signal and/or depth of read signal (e.g., used in combination) may be obtained from a sample of genetic material collected from the subject. The one or more signals may be obtained from cell-free DNA. The one or signals may be obtained from cellular DNA. If an allele balance signal is used, the true signal may be determined by correcting the allele balance signal using a non-error-propagating phasing technique, as described elsewhere herein. The non-error-propagating phasing technique may be performed on cellular DNA. According to some aspects in which the one or more signals indicative of ploidy status are obtained from cellular DNA and non-error-propagating phasing is performed on cellular DNA, the same source of cellular DNA may be used for both. The cellular DNA may be obtained from blood cells (e.g., white blood cells) or other cells collected via noninvasive or minimally invasive techniques. In some implementations, cell-free DNA for obtaining the genetic signals of ploidy status and cellular DNA for performing the non-error-propagating phasing are obtained from the same biological sample (e.g., blood draw). A ploidy status determination may be made from the one or more signals to evaluate the ploidy status of the assessed DNA. Allele balance and/or depth of read (e.g., used in combination) may be used to identify a difference in copy number between variants at the same locus, indicating an aneuploidy in one of the chromosomal homologues.

The methods described herein may be used to detect nherited variations in ploidy status (i.e. a variation in ploidy status at one or more loci of one of a subject's chromosomes, in which the ploidy status of each chromosomal homologue was inherited from a parent) or de novo variations in ploidy status (i.e. a change in ploidy status of one of a subject's chromosomes relative to the ploidy status in the corresponding chromosomal homologue or haplotype of the parent from which the chromosomal homologue or haplotype was inherited). The inherited haplotype can be used to provide a reference genetic code relative to which the ploidy status detected in the subject can be compared. If the aneuploidy is present in the genetic code of either of the parents then the aneuploidy can be determined to be inherited. If the aneuploidy is not present in the genetic code of either of the parents then the aneuploidy can be called as a de novo variation.

According to some aspects of the invention, a determination of the parent of origin of the haplotype having an aneuploidy status is made. Such determinations may be possible, for example, based on the phasing of the variant and the prior probability of maternal/paternal copy number. Additional sequencing may be performed on one (the originating parent) or both of the parents to confirm the determination. For example, whole genome sequencing (e.g., shotgun sequencing) may be performed on the parent(s), which may allow confirmation of the corresponding copy number in the originating parent.

According to specific aspects of the invention, the subject may be an embryo or a fetus. As used herein, an “embryo” may refer to a cellular organism produced by sexual reproduction, including a zygote, morula, and blastocyte, up to the stage of development where the embryo becomes a fetus. An embryo, may exist in vitro (e.g., for purposes of IVF) or in utero. As used herein, a “fetus” may refer to an unborn offspring produced by sexual reproduction and existing in utero, beginning at the stage of development where the unborn offspring is no longer characterized as an embryo. Thus, a subject may be considered either an embryo or a fetus from the single cellular stage until the fetus is born. In humans, the offspring is usually considered to be a fetus at approximately 8 weeks following conception. It is well understood in the art what types of genetic material can be effectively obtained from an embryo or a fetus as well as the techniques for doing so and any inherent risks associated therewith.

Determination of ploidy status for an embryo of fetus (including calls of de novo changes) may generally be performed as described elsewhere herein (e.g., for a born child or adult individual). However, de novo detection in unborn subjects may present certain challenges. For example, cellular DNA for performing non-error-propagating phasing may not be as readily available. For instance, collecting body fluid samples, such as blood samples containing circulating blood cells, may be impractical or impossible, depending on the stage of development. Furthermore, collecting cellular material, in general, from an embryo or fetus may pose risks to the viability or health of the subject (e.g., spontaneous abortion). According to some aspects, cellular DNA may be obtained from a biopsy of an embryo or fetus, as is known in the art. In preferred implementations of performing ploidy status determinations on an embryo or fetus, non-error propagating phasing may be performed on samples collected from one or more genetic relatives, for example the mother and/or father. Cellular DNA may be obtained, for example, from a body fluid (e.g., blood) sample or other tissue type obtained from the genetic relative(s) and used to correct the phasing of a reference genetic code, as described elsewhere herein. Cell-free DNA may be collected from the genetic relative(s) as needed. In some implementations, the reference genetic code may be constructed, at least in part, based on the sequencing of one or more genetic relatives (e.g., whole genome shotgun sequencing) as is known in the art. See, e.g., Kitzman et al., Sci Transl Med. 2012 Jun. 6; 4(137):137ra76 (doi: 10.1126/scitranslmed.3004323). For example, the analysis of the genetic relative's genome may identify variants for subsequent analysis in the subject. Cell-free DNA from an embryonic or fetal subject may be collected for analysis according to any suitable method known in the art. For example, cffDNA may be collected from the blood of a mother carrying the subject fetus or subject embryo, to the extent sufficiently developed. Cell-free DNA may be collected from the blastocele fluid of an embryo or from the cell culture medium used to culture an embryo for IVF as is known in the art. The cell-free DNA of the fetus or embryo may be used, at least in part, to determine the genome of the subject (e.g., via whole genome shotgun sequencing) and/or establish a reference genetic code for ploidy status calls. See, e.g., Kitzman et al., Sci Transl Med. 2012 Jun. 6; 4(137):137ra76 (doi: 10.1126/scitranslmed.3004323). Sequencing of the cell-free DNA may be used, at least in part, to phase the subject's genome or the reference genetic code (e.g., via molecular techniques known in the art). Sequences of one or more genetic relatives and/or population reference panels may be used in combination with the sequencing of the cell-free DNA to provide an at least partially phased genome (prior to any correction of phasing by non-error-propagating phasing techniques). The cell-free DNA collected from the embryonic or fetal subject may be used to generate an allele frequency signal and/or depth of read signal, as described elsewhere herein, from which ploidy status calls can be made. The allele frequency signal may be corrected using the non-error-propagating phasing techniques performed on the cellular DNA of the subject's one or more genetic relatives.

Examples of specific associations between aneuploidies (e.g., CNVs or whole chromosomal abnormalities) and disease are well known in the art. According to some aspects of the invention, the determination of ploidy status may be used to inform decisions on IVF. The methods described herein may be performed on a single embryo or on a plurality of embryos (e.g., a plurality of embryo candidates for implantation). The determination of ploidy status may be used to select one or more embryo's for implantation and/or to select one or more embryo's for discarding/disposal. The determination of ploidy status may be used to select one or more embryo's for freezing (either in the case that the embryo is selected for possible future implantation or in the case that the embryo is not a primary candidate for implantation but it is not desired to be disposed of). For example, a determination of risk of disease may be made for an embryo at least in part based on the detection of an aneuploid status for a chromosome or chromosomal segment (e.g., the identification of a CNV, particularly one having a known association with a disease). In some implementations, an embryo with no identified aneuploidies (e.g., CNVs) may be selected for implantation or freezing. In some implementations, the embryos may be ranked based entirely or at least in part on the identification of aneuploidies (e.g., by the number of CNVs and/or the presence of particular CNVs). The determination of ploidy status according to the methods described herein may be used independently or in combination with existing methods of preimplantation genetic testing (PGT), as is well known in the art.

According to some aspects of the invention, the determination of ploidy status may be used to inform decisions on pregnancy, particularly where the subject is a fetus. For example, the decision whether to continue or terminate a pregnancy may be based on the determination of ploidy status (e.g., the identification of an aneuploidy) in the same manner as decisions are made regarding IVF, as described elsewhere herein. The determination of ploidy status according to the methods described herein may be used independently or in combination with existing methods of prenatal diagnosis, as is well known in the art.

According to certain aspects of the invention, the determination of ploidy status may be used to inform additional testing and/or methods of diagnosis. For example, upon the identification of an aneuploidy, additional PGD or prenatal diagnostic testing may be ordered. In some instances, the additional testing may be specific to one or more diseases associated with an aneuploidy detected. In some instances, more invasive procedures may be performed on the subject, particularly if the subject is an embryo or fetus. For example, tissue biopsies may be performed directly on the embryo or fetus in order to perform sequencing of cellular DNA or other diagnostics on the cellular material. Karyotyping may be performed on the subject. In some implementations, the additional testing may be performed substantially concurrently with the determination of ploidy status (at approximately the same level of development). In some implementations, additional testing may be performed on a postponed schedule, allowing for additional development to occur (e.g., for development from an embryo to a fetus and/or after implantation of an embryo via IVF). In some implementations, additional testing may be performed on a born subject (e.g., an infant or child subject) based on ploidy status determinations made when the subject was an embryo and/or fetus.

According to certain aspects of the invention, the determination of ploidy status may be used to inform treatment decisions for the subject. For example, upon the identification of an aneuploidy, the subject may be treated for a disease or condition associated with the aneuploidy. The treatment may comprise any treatment suitable for the subject's stage of development. For example, genetic editing may be performed on an embryo and/or prenatal treatments may be administered to a fetus (or mother carrying the fetus). In some implementations, treatments may be performed on a postponed schedule, allowing for additional development to occur (e.g., for development from an embryo to a fetus and/or after implantation of an embryo via IVF). In some implementations, treatment may be performed on a born subject (e.g., an infant or child subject) based on ploidy status determinations made when the subject was an embryo and/or fetus. The early detection of an aneuploidy (e.g., while in utero) may allow for earlier treatment in infants and children, which may provide improved outcomes.

### Disease Diagnosis

In addition to diagnoses described elsewhere herein that are based on known associations of an aneuploidy (e.g., CNV) with a disease, the methods described herein may be used to identify novel associations between aneuploidies and diseases. By identifying the same aneuploidy among a population of subjects having a particular disease or disposition for a disease an association between the aneuploidy and disease may be established.

The use of phase determined by the non-error-propagating phasing of one or more rare aneuploid variants and the identification of neighboring SNPs known to be associated with a disease (e.g., within the same haplotype block or within two phase sets determined to be in phase alignment via the methods described herein), can be used to clarify the function of the SNP, particularly as relates to the disease. The rare variant and identified SNP may be determined to be in linkage disequilibrium. The rare variant can be effectively linked to the identified SNP by increasing the contribution to disease risk (e.g., in a polygenic risk score (PRS)) of that SNP, relative to other neighboring SNPs (e.g., that are in linkage disequilibrium with the identified SNP). The linkage of the rare variant to the more common SNP can, thus, improve the predictive power of the more common SNP as it relates to predisposition of the disease.

Upon identification of an aneuploidy variant associated with a disease, sequencing may be conducted in other subjects for diagnostic purposes of determining predisposition for the disease. The sequencing may be targeted to capture the aneuploidy variant. The sequencing may be conducted to target neighboring SNPs, such as those determined to be in linkage disequilibrium with the aneuploid variant, as described elsewhere herein, (e.g., via microarrays). The sequencing may be conducted to target both an aneuploidy variant (e.g. a rare variant) and SNP (e.g., a common SNP).

Diagnosis of a disease may be made based, at least in part, on the presence or absence of one or more aneuploid variants and/or based, at least in part, on one or more SNPs determined to be in linkage disequilibrium with the one or more aneuploid variants. Diagnosis may be made, for example, based on a PRS, as is well known in the art. Treatment for the disease may be informed based on any of the diagnostic methods described herein. For example, a subject may be treated (including prophylactic treatment) for a disease for which the subject has been diagnosed as having or at least having an increased disposition for having or developing. Diagnosis and treatment may be performed in combination with other clinical factors and variables as is understood in the art.

### Phasing Germline Mosaic Variants

The methods described herein may be used to identify a haplotype in an affected individual having an aneuploid variant. Gametes from the affected individual may be screened (e.g., to avoid gametes carrying the identified haplotype) for purposes of IVF.

According to certain aspects of the invention, the use of non-error-propagating phasing techniques can be applied to phase a germline mosaic variant in an affected individual. Such affected individuals may comprise, for example, an individual with Noonan Syndrome or RASopathy. This phased information can be used to inform decisions regarding IVF, as described elsewhere herein. For example, the phased information may be used to determine which haplotype to avoid in a subsequent generation using IVF and PGT.

According to certain aspects of the invention, long phased reads may be used to include the prediction of rare variants in the genome of an embryo by linking the rare variant to a common variant (e.g., SNP) in each of two parents and then subsequently inferring the inheritance of that rare variant in an embryo after determining which SNP was inherited in the embryo.

### EXAMPLES

### Example 1

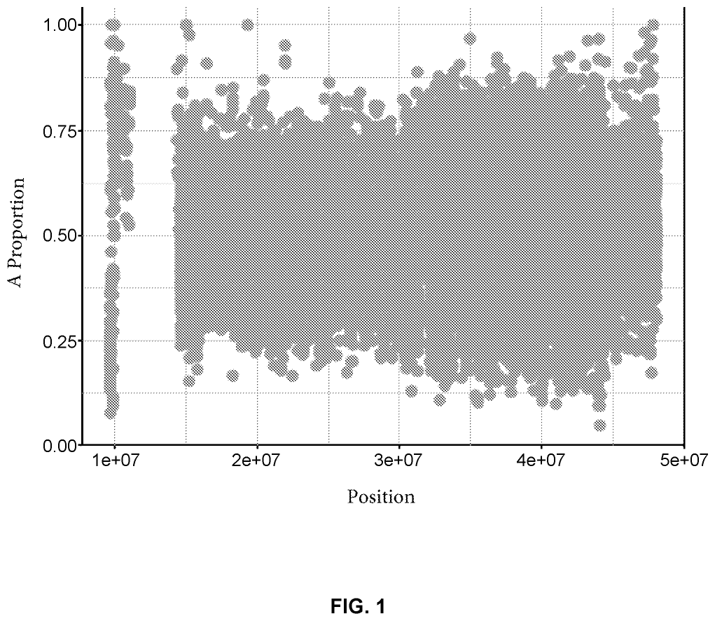

A dataset of synthetic reads corresponding to a specific haplotype was generated from a phased genome in order to simulate a chromosomal imbalance (amplification) on human chromosome 21. In brief, reads from nucleotide positions 30227447-44327015 of genetic sample NA12878 were added to data generated using a 10× GENOMICS® synthetic long read approach (CHROMIUM® product) according to the methods described in Samadian et al., PLoS Comput Biol. 2018 Mar. 28; 14(3):e1006080 (doi: 10.1371/journal.pcbi.1006080), which is herein incorporated by reference in its entirety. The inputs to this software included a phased VCF file, which includes a phase shift error at approximately the 37 Mb position, and a sequencing file (bam). 200,000 of these reads were then added to a set of standard shotgun reads obtained from the 1000 Genomes repository. Positions predicted to be “0|1” based on the Platinum Genomes variant set for sample NA12878 were assigned the “A” haplotype, and positions predicted to be “1|0” were assigned the “B” haplotype. See, e.g., Eberle et al., Genome Res. 2017 January; 27(1):157-164 (doi: which is herein incorporated by reference in its entirety. Positions were filtered for a depth>5 reads or depth>20 reads. Each position was assigned to an “A” allele or “B” allele based on the phasing of the inputted phased VCF file. FIG. 1 shows the allele balance, in terms of the proportion of A alleles, for heterozygous sites (SNPs) based on the dataset of synthetic reads for the chromosome.

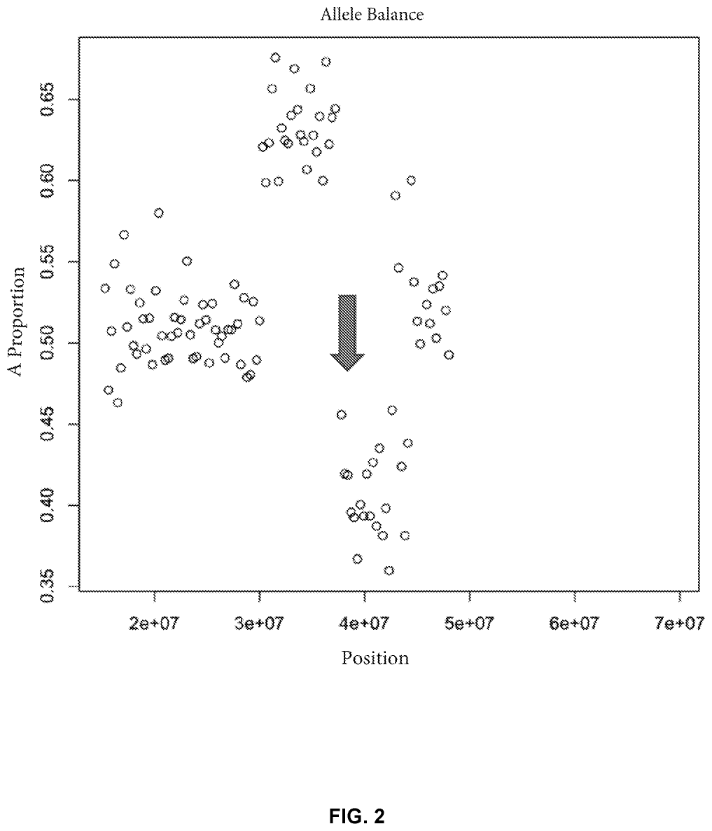

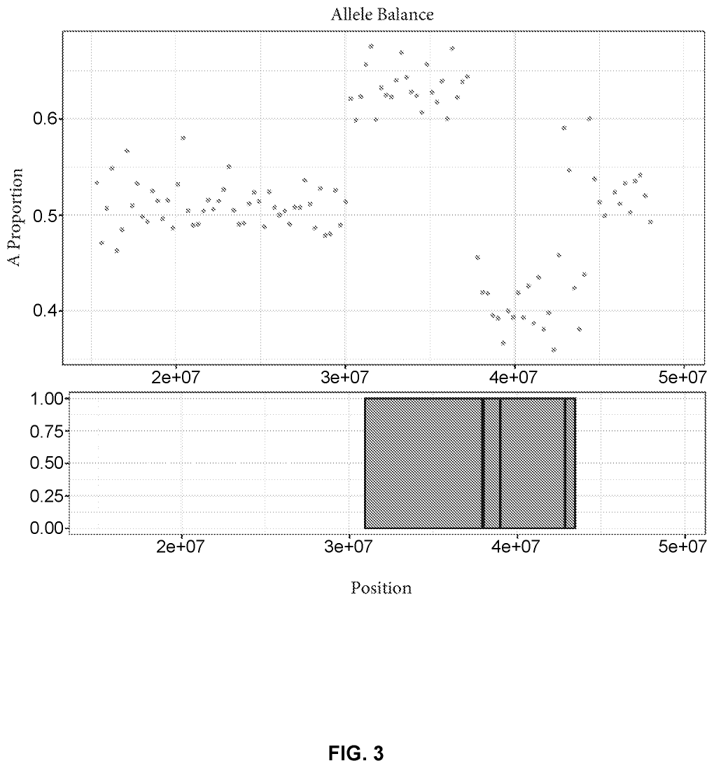

To improve the signal-to-noise ratio of the allele balance signal, consecutive SNPs on the same haplotype as determined by dilution pool sequencing were binned and the allele balance signal was averaged over the binned region, as shown in FIG. 2. In FIG. 3, the allele balance signal was averaged over 300 Kb windows of the haplotype blocks. As can be seen from the averaged allele balance signals in FIGS. 2 and 3, there appears to be two distinct aneuploidies —what could be a chromosomal amplification of the A haplotype, specifically a trisomy, from about position 30 Mb to position 37 Mb, immediately followed by a chromosomal deletion of the A haplotype, specifically a monosomy, from about position 37 Mb to position 44 Mb. The haplotype blocks determined from dilution pool sequencing over the aneuploidy region are depicted at the bottom of FIG. 3.

Data obtained from a Hi-C experiment for sample NA12878 was downloaded from staging.4dnucleome.org/filesprocessed/4DNFIY9YBG6F. The Hi-C data was able to be used to identify the switch error in the phased vcf and then correct the allele balance data in order to accurately call the aneuploidies, as described below. As the reference is hg38, vcf files were mapped to hg38. The tool “extractHAIRS” from the program HapCut2 was used to generate fragments of evidence supporting various combinations of phase blocks, as described in Edge et al., Genome Res. 2017 May; 27(5): 801-812 (doi: 10.1101/gr.213462.116), which is herein incorporated by reference in its entirety.

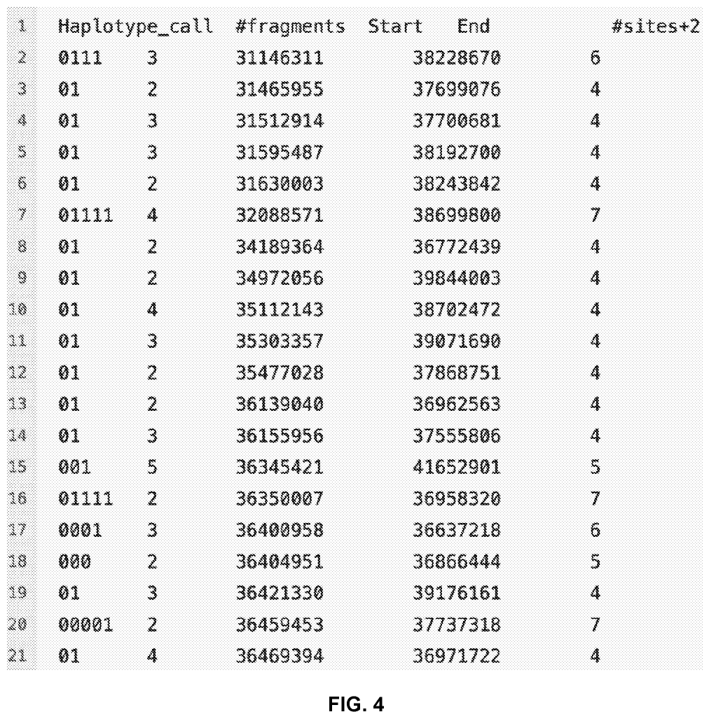

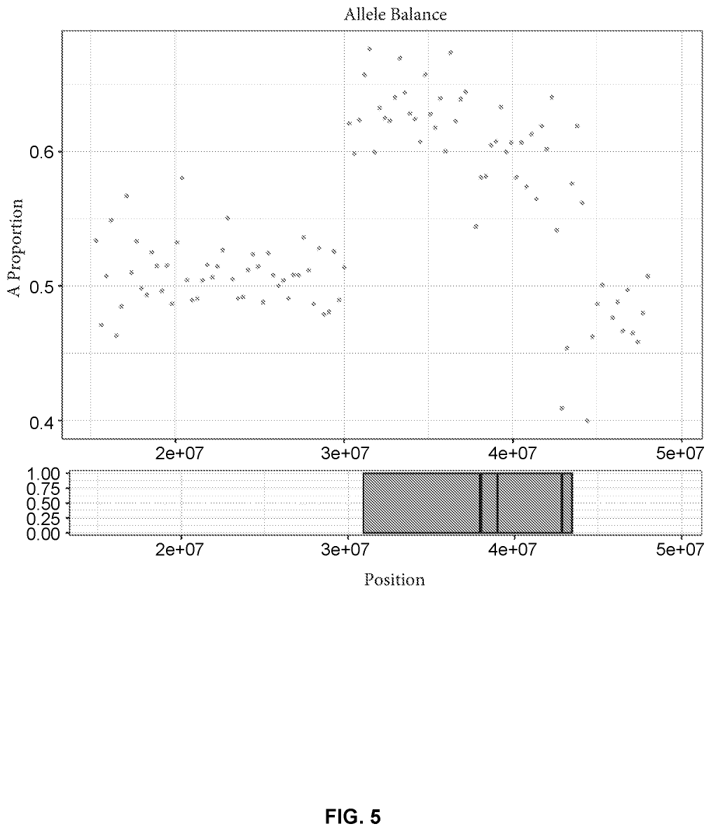

The phase alignment of two phase sets were evaluated using the Hi-C data. One phase set was defined as the set of SNPs existing approximately over the 30 Mb-37 Mb positions and the second phase set was defined as the remainder of the SNPs on chromosome 21 from approximately the 37 Mb position onward. Hi-C fragments containing informative reads (overlapping two or more heterozygous variants) are assembled into sparse sub-groups in which variants are self-consistent throughout the subgroup. Those sub-groups that at least partially overlap both of the phase sets (i.e. sub-groups having at least one SNP from each of the two phase sets) were further filtered from the Hi-C data and evaluated, as illustrated in FIG. 4, and the overlapping sub-groups were determined to be either entirely concordant (i.e. having no divergent haplotype calls, such as “00”, “000”, “0000”, etc.) or discordant (i.e. having at least one divergent haplotype call such as “01”, “011”, “0111”, etc.). The total number of sub-groups, including the distribution of entirely concordant and discordant fragments were tabulated. As shown in FIG. 4, there were 20 total subgroups, with 19 instances of discordance when compared to dilution pool sequencing and 1 instance of concordance with dilution pool sequences. The number of fragments refers to the number of fragment reads in each subgroup, wherein each fragment has at least two of the SNPs supporting the haplotype call, but not necessarily each of the SNPs in the subgroup. To evaluate the distribution of concordant and discordant measurements observed, the probability that the observed distribution would occur purely by chance, assuming an equal likelihood of obtaining a concordant measurement and a discordant measurement, was calculated using a binomial distribution. The binomial probability was very low, less than a 0.01% chance, that the skewed distribution would occur purely by chance. Thus, it was determined that the Hi-C measurements overlapping the two phase sets were predominantly discordant because the presumptive phase alignment between the two phase sets was in fact incorrect or misaligned. Assuming the phasing of the first phase set (approximately over the 30 Mb-37 Mb positions) to be correct and the phasing of the second phase set (from 37 Mb onward) to be incorrect by nature of a switch error introduced between the two phase sets, the phase of the second phase set was reversed and the true allele balance signal, averaged over the 300 Kb windows of haplotype blocks, corrected as shown in FIG. 5. The true allele balance signal shows a 14 Mb aneuploidy, approximately over positions 30 Mb to 44 Mb, which could theoretically correspond an amplification of haplotype A or a deletion of haplotype B.

### Example 2

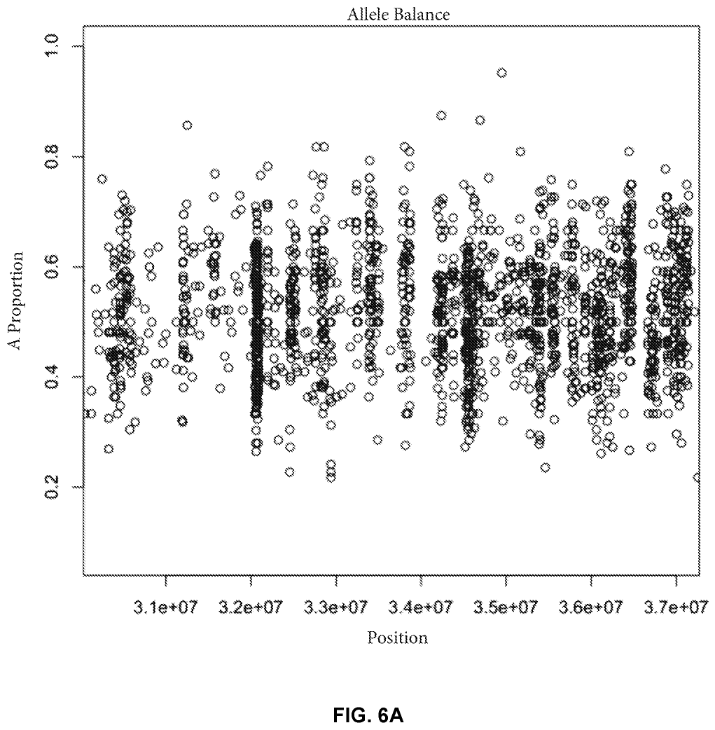

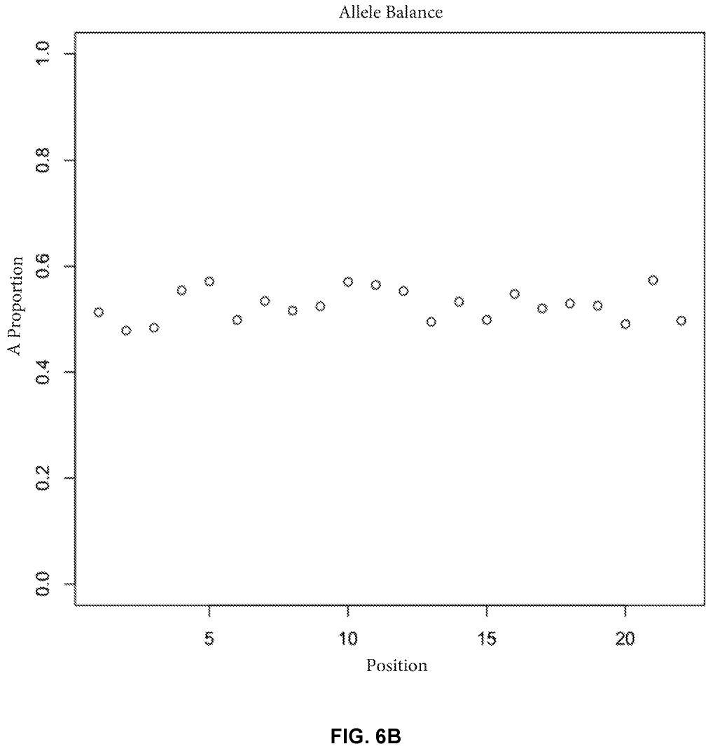

The simulated dataset of Example 1 was replicated, but with reads corresponding to the aneuploidy (amplification of haplotype A) in chromosome 21 being down-sampled to approximately 9% of the measured cells, with approximately 91% of the cells displaying euploidy over the same chromosomal segment. FIG. 6A shows the raw allele balance signal for the 30.3 Mb-37 Mb portion of the chromosome for heterozygous loci (SNPs). The allele balance signal over this range has a mean of 0.5232 and a standard deviation of 0.1141. FIG. 6B shows the same allele balance signal averaged over 300 Kb windows of haplotype blocks determined by dilution pool sequencing. As can be seen in FIG. 6B, the allele balance shift introduced by the 9% aneuploid cells is more readily discernible and the standard deviation has decreased to 0.0258 as a result of the binning. This example, thus, demonstrates the ability to call the amplification even at low allele fraction.

### Example 3

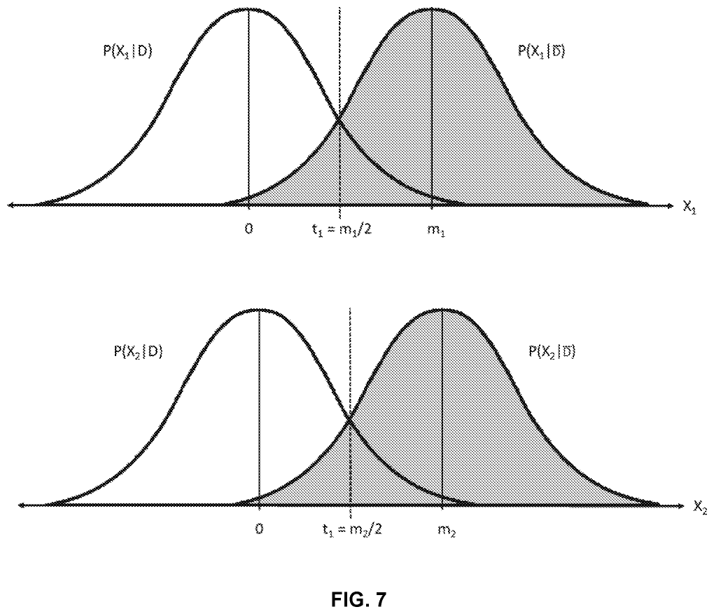

In this example, it was assumed that a population of disomy (D) measurements and a population of trisomy (D̅) measurements possessed normal distributions having equal standard deviations for a depth of read signal X₁, as schematically illustrated in FIG. 7. The mean of the trisomy population was offset against the mean of the disomy population, such that the disomy population has an effective mean of 0 and the mean of the trisomy population has an effective mean of m₁. Accordingly, the probability of a disomy or trisomy given a depth of read signal X₁ can be defined as follows:

$$P ( D \vert {X}_{1} ) = \frac{P ( {X}_{1} \vert D ) P ( D )}{P ( {X}_{1} )} , and$$

$$P ( \overline{D} \vert {X}_{1} ) = \frac{P ( {X}_{1} \vert \overline{D} ) P ( \overline{D} )}{P ( {X}_{1} )} .$$

It was assumed that the overall probability of disomy is equal to the overall probability of trisomy (i.e., P(D)=P(D̅)). The threshold, t₁, above which a depth of read signal X₁ is considered to be indicative of trisomy was set at the X₁ level of m₁/2, where the probability of trisomy is equal to the probability of disomy for the same X₁ signal (i.e., P(D̅|X₁)=P(D|X₁)). Accordingly, the above equations can be solved to show that at t₁:

P(X₁|D̅)=P(X₁|D)).

The probability of the signal X₁ corresponding to a false positive (i.e. falsely characterizing disomy as trisomy) was then computed from the cumulative distribution function using X₁ as follows:

$$Probability of false positive = {\int }_{P ( {X}_{1} \vert D ) < P ( {X}_{1} \vert \overline{D} )}^{inf} P ( {X}_{1} \vert D ) {dx}_{1}$$

$$= {\int }_{t}^{inf} P ( {X}_{1} \vert D ) {dt}_{1}$$

$$= {\int }_{\frac{m 1}{2}}^{inf} \frac{1}{sqrt ( 2 * pi )} {e}^{\frac{- {X}_{1}^{2}}{2}} {dx}_{1}$$

A method of making a disomy/trisomy call from using two signals together—X₁, a depth of read signal and an orthogonal signal, X₂ (e.g., an allele balance signal)—was computationally simulated according to the call scheme shown in Table 1, below:

TABLE 1

| X₁ | X₂ | Combined call |
|---|---|---|
| <m₁/2 | <m₂/2 | disomic |
| >m₁/2 | >m₂/2 | trisomic |
| <m₁/2 | >m₂/2 | No call |
| >m₁/2 | <m₂/2 | No call |

The same assumptions made for the distribution of signal X₁, as described above, were made for the distribution for signal X₂. The probability of calling a false positive and the probability of failing to make any call based on the using both distributions according to Table 1 were determined as follows in Table 2, wherein “normcdf” is the normal cumulative distribution function (e.g., as in MATLAB®):

TABLE 2

| Probability | Variable | Calculation |
|---|---|---|
| Probability of False | P_{FPX1} | 1-normcdf(m₁/2) |
| Positive based on X₁ | | |
| Probability of False | P_{FPX2} | 1-normcdf(m₂/2) |
| Positive based on X₂ | | |
| Probability of False Positive | P_{FPX1X2} | P_{FPX1} * P_{FPX2} |
| based on X₁ and X₂ | | |
| Probability of No Call based | P_{NCX1X2} | P_{FPX1}*(1-P_{FPX2}) + |
| on X₁ and X₂ | | P_{FPX2}*(1-P_{FPX1}) |

Assuming m₁=6 and m₂=6/sqrt(3), probability values were calculated as follows: P_{FPX1}=0.0013; P_{FPX2}=0.0416; and P_{FPX1X2}=0.000056.

### Example 4

A population of disomy (D) measurements and a population of trisomy (D̅) measurements were assumed to have the same distributions as in Example 3. A method of making a disomy/trisomy call from mathematically combining the two signals, X₁ and X₂, into a single product (X₁*X₂ or “X₁X₂”) was calculated as follows:

$$P ( D \vert {X}_{1} {X}_{2} ) = \frac{P ( {X}_{1} {X}_{2} \vert D ) P ( D )}{P ( {X}_{1} {X}_{2} )} ,$$

$$and$$

$$P ( \overline{D} \vert {X}_{1} {X}_{2} ) = \frac{P ( {X}_{1} {X}_{2} \vert \overline{D} ) P ( \overline{D} )}{P ( {X}_{1} {X}_{2} )} .$$

Assuming again that the overall probability of disomy is equal to the overall probability of trisomy (i.e., P(D)=P(D̅)), then at threshold, t:

P(D|X₁X₂)=P(D̅|X₁X₂),

P(D|X₁X₂)=P(X₁|D)P(X₂|D), and

P(D̅|X₁X₂)=P(X₁|D̅)P(X₂|D).

The joint probability function was then integrated to evaluate the false positive rate, as follows:

$$P ( False Positive ) = {\int }_{P ( {X}_{1} \vert D ) P ( {X}_{2} \vert D ) < P ( {X}_{1} \vert \overline{D} ) P ( {X}_{2} \vert \overline{D} )}^{inf} P ( {X}_{1} \vert D ) P ( {X}_{2} \vert D ) {dx}_{1} {dx}_{2}$$

$$P ( False Positive ) = {\int }_{P ( {X}_{1} \vert D ) P ( {X}_{2} \vert D ) < P ( {X}_{1} \vert \overline{D} ) P ( {X}_{2} \vert \overline{D} )}^{inf} \frac{1}{2 * pi} {e}^{\frac{- ( {X}_{1}^{2} + {X}_{2}^{2} )}{2}} {dx}_{1} {dx}_{2}$$

where P(X₁|D)P(X₂|D)<P(X₁|D̅)P(X₂|D̅) can be clarified further to:

$$\frac{P ( {X}_{1} \vert D ) P ( {X}_{2} \vert D )}{P ( {X}_{1} \vert \overline{D} ) P ( {X}_{2} \vert \overline{D} )} < 1$$

$${e}^{\frac{- ( {X}_{1}^{2} )}{2 {σ}^{2}} + \frac{{( {X}_{1} - {m}_{1} )}^{2}}{2 {σ}^{2}} *} {e}^{\frac{- ( {X}_{2}^{2} )}{2 {σ}^{2}} + \frac{{( {X}_{2} - {m}_{2} )}^{2}}{2 {σ}^{2}}} < 1$$

$$2 {X}_{1} {m}_{1} + 2 {X}_{2} {m}_{2} > {m}_{1}^{2} + {m}_{2}^{2} .$$

X₂ was then solved as follows:

$${X}_{2} > \frac{{m}_{1}^{2} + {m}_{2}^{2} - 2 {m}_{1} {X}_{1}}{2 {m}_{2}} .$$

Accordingly, the false positive rate was determined to be:

$$P ( False Positive ) = {\int }_{- inf}^{inf} {\int }_{\frac{{m}_{1}^{2} + {m}_{2}^{2} - 2 {m}_{1} {X}_{1}}{2 {m}_{2}}}^{inf} \frac{1}{2 * pi} {e}^{\frac{- ( {X}_{1}^{2} + {X}_{2}^{2} )}{2}} {dx}_{1} {dx}_{2}$$

The false positive rate was then able to be empirically calculated using the following MATLAB® code, wherein “sum” is the false positive rate, for different signal means, m₁ and m₂:

```
% variables
n = 2000;
m1 = 6;
m2 = 6/sqrt(3);
lim = 20;
delta = 2*lim/(n−1);
x1_vec = [−lim:delta:lim];
x2_vec = [−lim:delta:lim];
sum = 0;
for x1 = x1_vec
  ind = find( x2_vec > (m1\hat{ }2 + m2\hat{ }2 −2*m1*x1)/(2*m2) );
  for x2 = x2_vec(ind)
    sum = sum + exp(−0.5*(x1\hat{ }2+x2\hat{ }2))*delta\hat{ }2/(2*pi);
  end
end
sum
```

The simulation was conducted using the same signal means as in Example 3. Here, “sum” corresponds to probability of observing a false positive in this joint probability scenario combining signal mean m1 and the somewhat weaker signal mean m2. The probability of a false positive was determined to be: P(False Positive)=sum=0.00026, whereas the individual probabilities (evaluated in Example 3) were determined to be higher: P_{FPX1}=0.0013 and P_{FPX2}=0.0416.

The simulation demonstrates that combining two independent signals, with one signal having a 3-fold higher variance than the other, can reduce the false positive rate by at least a factor of 5, relative to using either of the signals alone.

### Example 5

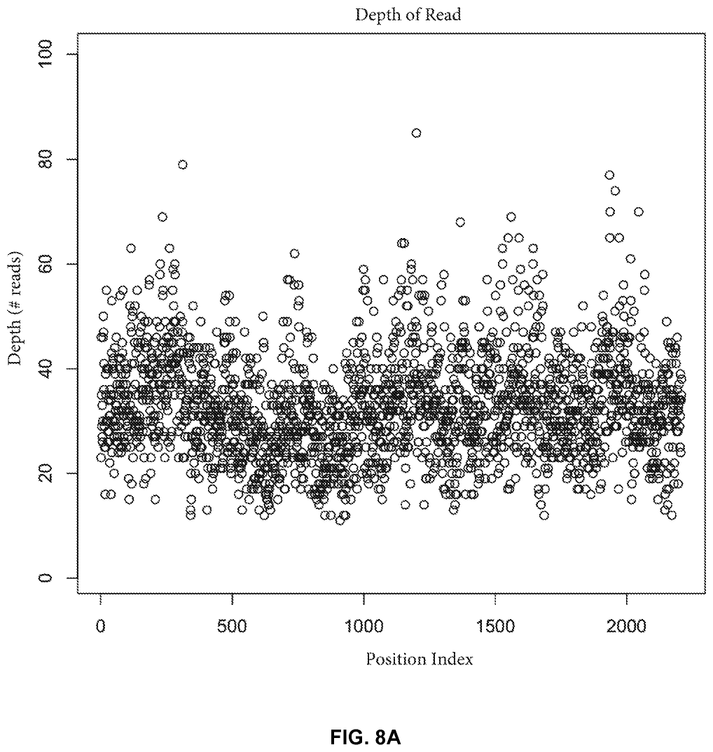

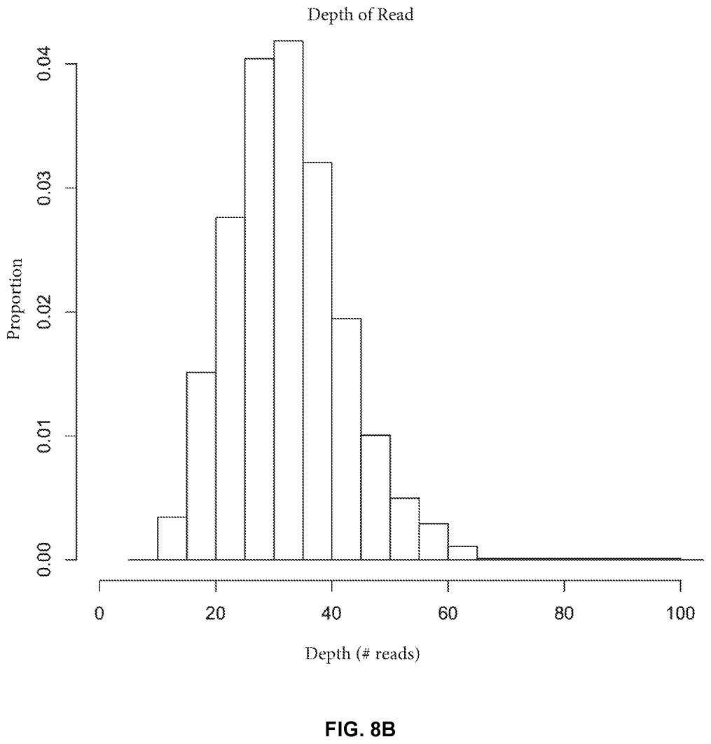

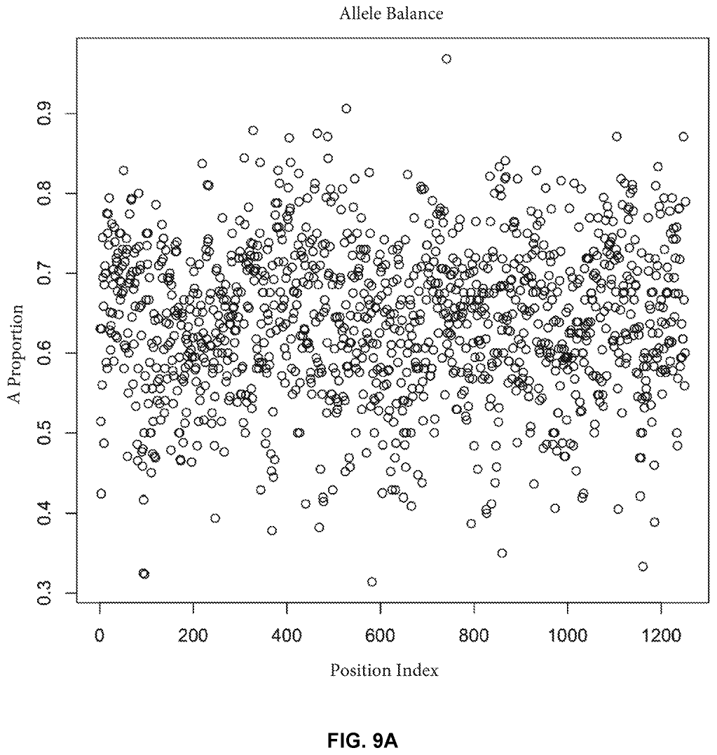

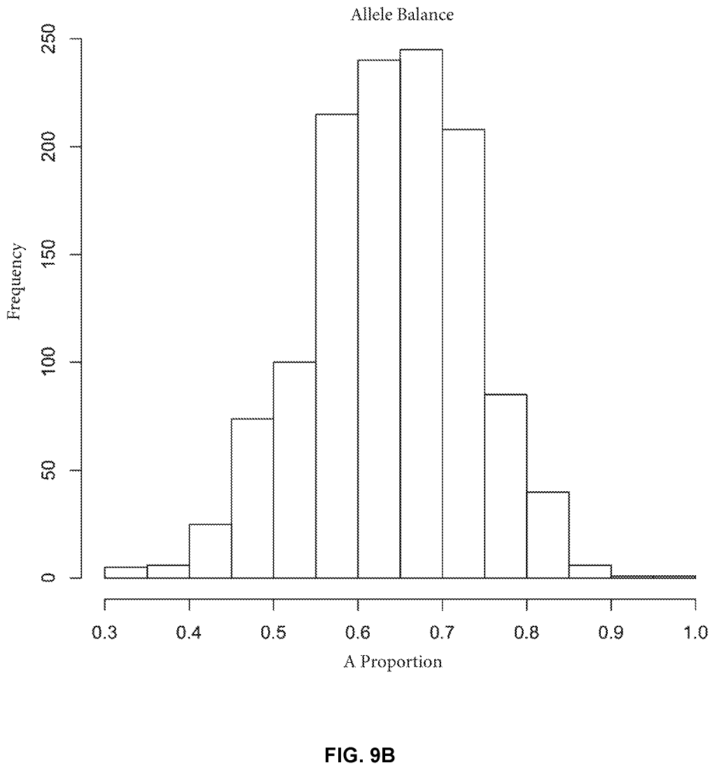

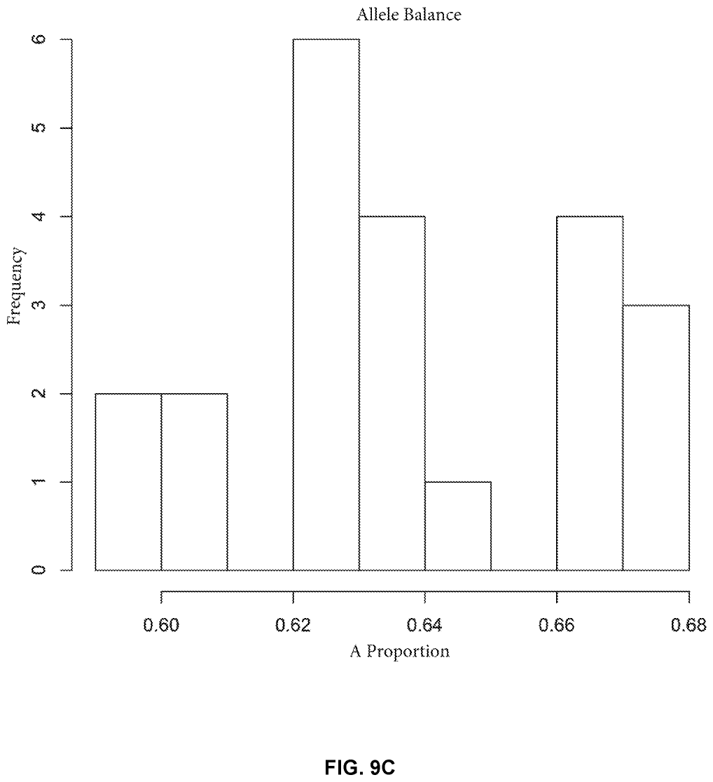

In a similar manner as Example 1, a synthetic aneuploid mixture of DNA was created with an amplification starting from position 30.3 Mb on chromosome 21. FIG. 8A shows the depth of read signal for positions between 31 Mb an 37 Mb, and FIG. 8B depicts a histogram of the binned depth of read measurements for positions between 31 Mb an 37 Mb. Similarly, FIG. 9A shows the allele balance signal for positions between 31 Mb and 37 Mb, and FIG. 9B depicts a histogram of the binned allele balance measurements for positions between 31 Mb an 37 Mb. FIG. 9C shows a histogram of binned allele balance measurements where measurements were averaged across 50 neighboring SNPs.

The mean signal-to-noise was calculated from the aggregated data, as described in U.S. Pat. No. 8,682,592 to Rabinowitz et al., issued on Mar. 25, 2014, which is herein incorporated by reference in its entirety. As was described in the theoretical simulations of Examples 3 and 4, threshold signal values for indicating trisomy were selected to be halfway between a mean diploid signal and a mean triploid signal for both depth of read and allele balance, approximating the scenario where the probability of calling a false negative is equal to the probability of calling a false positive as in Examples 3 and 4, although other thresholds could be selected. The mean signals for diploidy were determined by calculating the mean measurements over positions between 20 Mb and 30.3 Mb and the mean signals for triploidy were determined by calculating the mean measurements over positions between 30.3 Mb and 37 Mb. The threshold values were accordingly determined to be 31.5 reads per position and 58% A (0.58) for depth of read and allele balance signals, respectively.

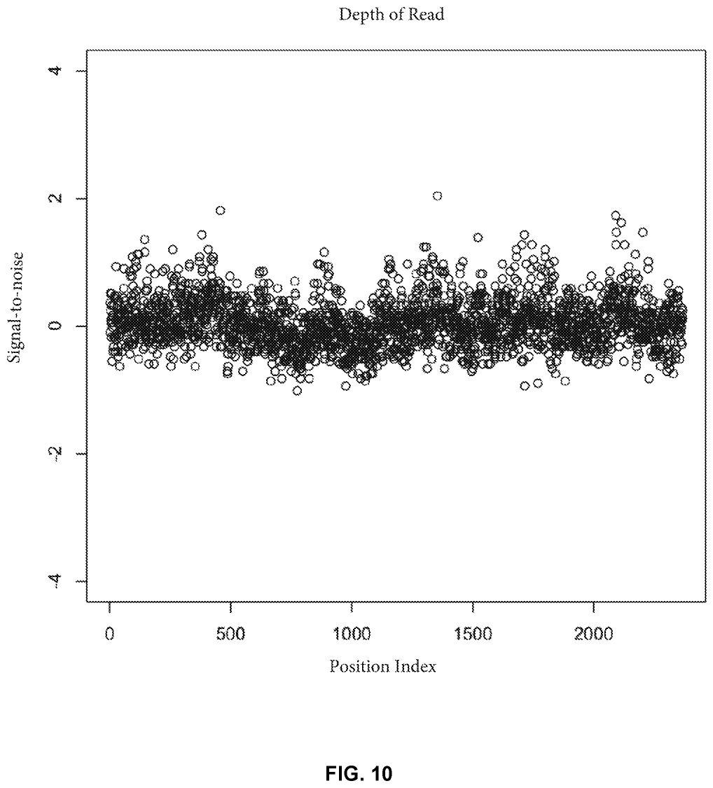

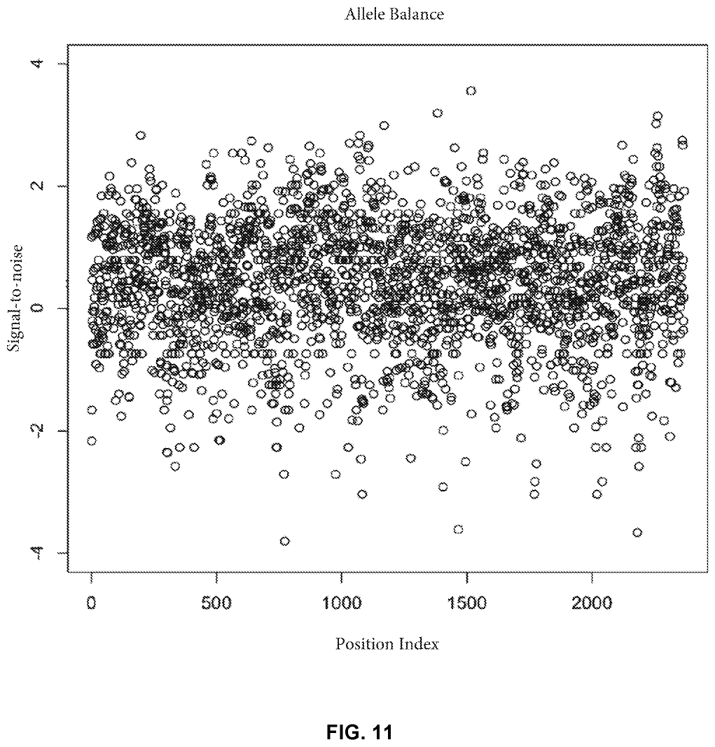

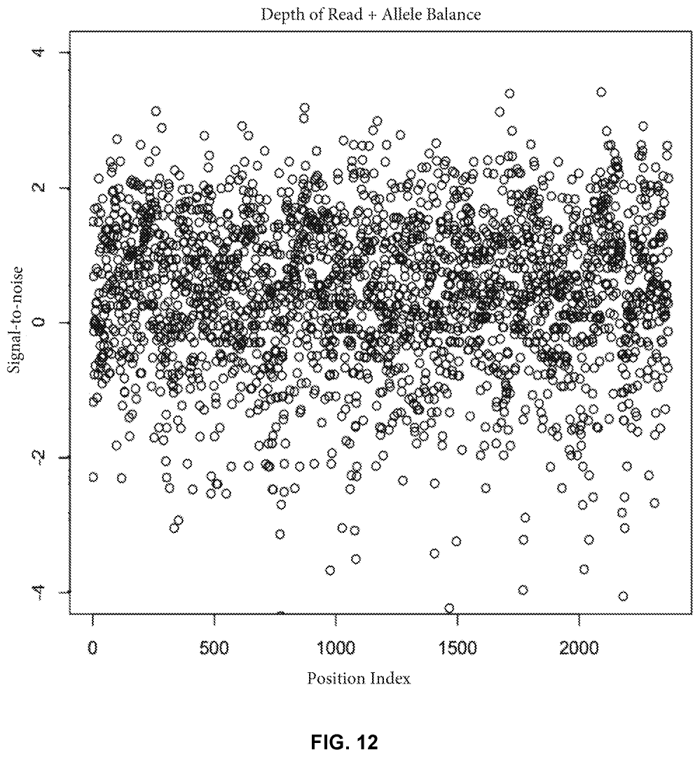

Signal-to-noise plots were generated for the depth of read signal and allele balance signal over the approximately 2500 measurements/positions of the amplification by subtracting the corresponding threshold value from the signal value at each position and then normalizing to the level of noise by dividing by the standard deviation measured over the region of the amplification. FIG. 10 shows the signal-to-noise plot for the depth of read signal and FIG. 11 shows the signal-to-noise plot for the allele balance signal. FIG. 12 shows the combined signal resulting from adding the signal-to-noise values for depth of read and allele balance together. The mean and standard deviation for the combined signal shown in FIG. 12 was calculated to be 0.4940 and 0.11, respectively.

While the invention has been described and exemplified in sufficient detail for those skilled in this art to make and use it, various alternatives, modifications, and improvements should be apparent without departing from the spirit and scope of the invention. The examples provided herein are representative of preferred aspects, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention. Various aspects of the invention will be understood to be combinable unless not physically possible or otherwise indicated by context.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred aspects and optional features, modification and variation of the concepts herein disclosed may be resorted to by those skilled in the art, and that such modifications and variations are considered to be within the scope of this invention as defined by the appended claims.

What is claimed is:

1. A method of correcting an allele balance signal for a chromosomal segment, the method comprising:
obtaining a reference genetic code comprising two phase sets, each phase set having one or more variants of interest, optionally wherein the reference genetic code is at least partially phased;
obtaining the allele balance signal for the one or more variants of interest from sequencing performed on a sample of genetic material;
obtaining a plurality of reads sequenced using a non-error-propagating technique, wherein each read comprises at least one of the one or more variants of interest;
determining the phase alignment of the two phase sets as being in phase or out of phase based on the plurality of reads; and
determining a true allele balance signal by confirming, correcting, or supplying the phasing of at least one variant of interest based on the determined phase alignment of the two phase sets.

2. The method of claim 1, wherein the non-error-propagating technique comprises chromosome conformation capture, single-cell template strand sequencing, or chromosomal isolation (e.g., via laser capture microdissection or karyotype).

3. The method of claim 1 or 2, further comprising performing the non-error-propagating technique to obtain the plurality of reads.

4. The method of any one of the preceding claims, wherein obtaining the allele balance signal comprises performing the sequencing on the sample of genetic material.

5. The method of any one of the preceding claims, wherein the allele balance signal and the plurality of reads are derived from the same sample of genetic material, optionally wherein the sample is a body fluid sample (e.g., a blood sample, a saliva sample) or a tissue biopsy sample, further optionally wherein the allele balance signal and the plurality of reads are derived from a same population of cells.

6. The method of any one of the preceding claims, wherein the allele balance signal is derived from cell-free DNA and the plurality of reads are derived from cellular DNA, optionally wherein the cellular DNA is from cells found within a body fluid (e.g., blood or saliva).

7. The method of any one of the preceding claims, wherein the reference genetic code is derived from the sequencing used to generate the allele balance signal.

8. The method of any one of the preceding claims, wherein the reference genetic code is derived, at least in part, from sequencing of normal tissue in a subject for which the allele balance signal is obtained.

9. The method of any one of the preceding claims, wherein the reference genetic code is derived, at least in part, from sequencing of germline tissue in a subject for which the allele balance signal is obtained.

10. The method of any one of the preceding claims, wherein the reference genetic code is derived, at least in part, from sequencing genetic material from one or more genetic relatives of a subject for which the allele balance signal is obtained.

11. The method of claim 10, wherein the one or more relatives is a mother and/or a father.

12. The method of claim 10 or 11, wherein the reference genetic code is derived, at least in part, from germline sequencing of the one or genetic relatives.

13. The method of any one of the preceding claims, wherein the reference genetic code is derived, at least in part, from whole genome shotgun sequencing of a subject for which the allele balance signal is obtained.

14. The method of claim 13, wherein the allele balance signal is derived from the whole genome shotgun sequencing.

15. The method of claim 13 or 14, wherein the whole genome shotgun sequencing is performed on cell-free DNA in a body fluid sample (e.g., a blood sample or saliva sample).

16. The method of any one of the preceding claims, wherein the non-error-propagating technique comprises single cell sequencing.

17. The method of any one of the preceding claims, further comprising collecting a sample of genetic material from which the allele balance signal is derived.

18. The method of any one of the preceding claims, further comprising collecting a sample of genetic material from which the plurality of reads are derived.

19. The method of any one of the preceding claims, wherein correcting the allele balance data comprises correcting a switch error in the at least partially phased reference genetic code.

20. The method of any one of the preceding claims, wherein the allele balance signal is averaged over a plurality of binned variants within a region of at least about 50,000, 100,000, 200,000, 300,000, 400,000, 500,000, 750,000, 1,000,000, 50,000,000, or 100,000,000 bp.

21. The method of any one of the preceding claims, wherein the allele balance signal is averaged over a plurality of binned variants within a region of no greater than about 50,000, 100,000, 200,000, 300,000, 400,000, 500,000, 750,000, 1,000,000, 50,000,000, or 100,000,000 bp.

22. The method of any one of the preceding claims, wherein the allele balance is averaged over a haplotype block.

23. The method of claim 22, wherein the haplotype block was determined by dilution pool sequencing, optionally wherein the allele balance signal was derived from the same sequencing.

24. The method of any one of the preceding claims, wherein the allele balance signal is filtered for a minimum read depth, optionally wherein the minimum read depth is 5, 10, 15, 20, or 25 reads.

25. The method of any one of the preceding claims, wherein the two phase sets are neighboring phase sets within the reference genetic code.

26. The method of claim 25, wherein each of the neighboring phase sets comprises a variant of interest which is no further than about 1,000, 5,000, 10,000, 50,000, 100,000, 500,0000, 1,000,000, 5,000,000, 10,000,000, 50,000,000, 100,000,000, or 250,000,000 bp from a variant of interest in the other.

27. The method of any one of the preceding claims, wherein the plurality of reads are filtered for reads comprising at least 2, 3, 4, or 5 of the variants of interest from each of the two phase sets.

28. The method of claim 2, wherein the non-error-propagating technique comprises chromosome conformation capture, optionally wherein the chromosome conformation capture is Hi-C.

29. The method of claim 28, wherein determining the phase alignment based on the plurality of reads comprises determining whether most of the reads are concordant or discordant with respect to a presumed phasing alignment between the two phase sets, optionally wherein the presumed phasing alignment is based on the at least partial phasing of the reference genetic code.

30. The method of claim 28 or 29, wherein determining the phase alignment based on the plurality of reads comprises determining or estimating a probability that an amount of concordance or discordance observed between the two phase sets from the plurality of reads is the result of chance.

31. The method of claim 30, wherein the probability is a binomial probability, optionally assuming that there is an equal chance than an observed fragment will be concordant or discordant.

32. The method of any one of the preceding claims, further comprising using the corrected allele balance signal to determine a ploidy status for a chromosomal segment, optionally wherein determining the ploidy status comprises calling a copy number variant (CNV).

33. A method of determining a ploidy status for a chromosomal segment, the method comprising:
obtaining a depth of read signal for a first set of one or more variants within the chromosomal segment;
obtaining an allele balance signal for a second set of one or more variants within the chromosomal segment; and
using the depth of read signal in combination with the allele balance signal to determine the ploidy status of the chromosomal segment.

34. The method of claim 33, wherein determining the ploidy status of the chromosomal segment comprises determining whether or not a CNV exists within the chromosomal segment.

35. The method of claim 33 or 34, wherein obtaining the depth of read signal comprises obtaining a number of sequencing reads mapped to at least one of the variants within the first set normalized relative to a total number of reads.

36. The method of any one of claims 33-35, wherein the depth of read signal is averaged over a binned plurality of variants within a region of at least about 50,000, 100,000, 200,000, 300,000, 400,000, 500,000, 750,000, 1,000,000, 50,000,000, or 100,000,000 bp.

37. The method of any one of claims 33-36, wherein the depth of read signal is averaged over a binned plurality of variants within a region no greater than about 50,000, 100,000, 200,000, 300,000, 400,000, 500,000, 750,000, 1,000,000, 50,000,000, or 100,000,000 bp.

38. The method of any one of claims 33-37, wherein the depth of read signal is averaged over a haplotype block.

39. The method of claim 38, wherein the haplotype block was determined by dilution pool sequencing.

40. The method of any one of claims 33-38, wherein the allele balance signal is averaged over a binned plurality of variants within a region of at least about 50,000, 100,000, 200,000, 300,000, 400,000, 500,000, 750,000, 1,000,000, 50,000,000, or 100,000,000 bp.

41. The method of any one of claims 33-40, wherein the allele balance signal is averaged over a binned plurality of variants within a region no greater than about 50,000, 100,000, 200,000, 300,000, 400,000, 500,000, 750,000, 1,000,000, 50,000,000, or 100,000,000 bp.

42. The method of any one of claims 33-41, wherein the allele balance signal is averaged over a haplotype block.

43. The method of claim 42, wherein the haplotype block was determined by dilution pool sequencing.

44. The method of any one of claims 33-43, wherein the depth of read signal and the allele balance signal are averaged over the same binned region.

45. The method of any one of claims 33-44, wherein using the depth of read signal in combination with the allele balance signal comprises making a positive or negative determination only when both the depth of read signal exceeds a depth of read threshold and the allele balance signal exceeds an allele balance threshold or when neither the depth of read signal exceeds the depth of read threshold nor the allele balance signal exceeds the allele balance threshold.

46. The method of any one of claims 33-44, wherein using the depth of read signal in combination with the allele balance signal comprises combining the depth of read signal and the allele balance signal into a single combined signal.

47. The method of claim 46, wherein combining the depth of read signal and the allele balance signal into a single combined signal comprises multiplying the signals together.

48. The method of claim 46, wherein combining the depth of read signal and the allele balance signal into a single combined signal comprises adding the signals together.

49. The method of any one of claims 46-48, wherein the combined signal is averaged over a binned plurality of variants within a region of at least about 50,000, 100,000, 200,000, 300,000, 400,000, 500,000, 750,000, 1,000,000, 50,000,000, or 100,000,000 bp.

50. The method of any one of claims 46-49, wherein the combined signal is averaged over a binned plurality of variants within a region no greater than about 50,000, 100,000, 200,000, 300,000, 400,000, 500,000, 750,000, 1,000,000, 50,000,000, or 100,000,000 bp.

51. The method of any one of claims 46-50, wherein the combined signal is averaged over a haplotype block.

52. The method of claim 51, wherein the haplotype block was determined by dilution pool sequencing.

53. The method of any one of claims 46-52, wherein the combined signal is averaged over a plurality of bins across which the depth of read signal and/or the allele balance signal were averaged.

54. The method of any one of claims 33-53, wherein the first set of one or more variants consists of 1 variant.

55. The method of any one of claims 33-53, wherein the first set of one or more variants comprises at least 2, 3, 4, 5, 10, 20, 30, 40, 50, 60, 70, 80, 90, 100, 200, 300, 400, 500, 600, 700, 800, 900, or 1,000 variants.

56. The method of any one of claims 33-55, wherein the second set of one or more variants consists of 1 variant.

57. The method of any one of claims 33-53, wherein the second set of one or more variants comprises at least 2, 3, 4, 5, 10, 20, 30, 40, 50, 60, 70, 80, 90, 100, 200, 300, 400, 500, 600, 700, 800, 900, or 1,000 variants.

58. The method of any one of claims 33-57, wherein the first set of one or more variants is identical to the second set of one or more variants.

59. The method of any one of claims 33-58, wherein obtaining the depth of read signal and/or obtaining the allele balance signal comprises performing sequencing.

60. The method of any one of claims 33-59, wherein the depth of read signal and allele balance signal are derived from the same sequencing data.

61. The method of any one of claims 33-60, wherein the depth of read signal and/or the allele balance signal is filtered for a minimum read depth, optionally wherein the minimum read depth is 5, 10, 15, 20, or 25 reads.

62. The method of any one of claims 33-61, further comprising calculating an individual probability of accurate determination of ploidy status based on the depth of read signal and/or the allele balance signal or calculating a joint probability of accurate determination of ploidy status based on the depth of read signal and the allele balance signal, optionally wherein the probabilities measure the probability of one of the following: a true positive, a false positive, a true negative, and a false negative.

63. The method of claim 62, wherein at least one of the following is true:
a) the joint probability of a false positive is less than both of the individual probabilities of a false positive;
b) the joint probability of a false negative is less than both of the individual probabilities of a false negative;
c) the joint probability of a true positive is greater than both of the individual probabilities of a true positive; and
d) the joint probability of a true negative is greater than both of the individual probabilities of a true negative;

64. The method of any one of claims 33-63, wherein the depth of read signal is offset against a first baseline signal and/or the allele balance signal is offset against a second baseline signal.

65. The method of claim 64, wherein each baseline signal is based on a mean signal for a second chromosomal segment having a known ploidy status, optionally wherein the second chromosomal segment having a known ploidy status is within the same chromosome as the chromosomal segment for which the ploidy status is being determined.

66. The method of any one of claims 33-65, wherein the depth of read signal and/or the allele balance signal is normalized against a measure of noise within the signal, optionally wherein the measure of noise is the standard deviation or variance of the signal over the chromosomal segment for which the ploidy status is being determined, over the second chromosomal segment of claim 65, over a third chromosomal segment having a known ploidy status of interest that is different from the ploidy status of the second chromosomal segment, or over the entire chromosome.

67. The method any one of claims 33-66, wherein a variance in the depth of read signal and a variance within the allele balance signal are within 100, 90, 80, 70, 60, 50, 40, 30, 10, 9, 8, 7, 6, 5, 4, 3, 2, 1.9, 1.8, 1.7, 1.6, 1.5, 1.4, 1.3, 1.2, or 1.1 fold of each other.

68. The method of any one of claims 33-67, wherein using the depth of read signal in combination with the allele balance signal results in reducing the false positive rate and/or the false negative rate by at least about 2, 3, 4, 5, 6, 7, 8, 9, 10, 20, 30, 40, 50, 60 70, 80, 90, 100, 150, 200, 250, or 500 fold relative to the false positive rate and/or false negative rate obtained with using one or both of the signals individually.

69. The method of any one of claims 33-68, wherein using the depth of read signal in combination with the allele balance signal comprises selecting a depth of read threshold and an allele balance threshold, optionally wherein the signal thresholds are each half the mean value of respective signals averaged over a plurality of variants known to exhibit a ploidy status of interest (e.g., an aneuploidy).

70. The method of any one of claims 33-69, wherein using the depth of read signal in combination with the allele balance signal comprises selecting a combined signal threshold, optionally wherein the combined signal threshold is half the mean value of a combined signal averaged over a plurality of variants known to exhibit a ploidy status of interest (e.g., an aneuploidy).

71. The method of any one of claims 33-70, wherein the method results in an aneuploidy of one or more chromosomes being detected.

72. The method of any one of claims 33-70, wherein the method results in euploidy of all chromosomes analyzed being detected.

73. The method of any one of claims 33-72, wherein the method results in an addition and/or deletion of a chromosomal segment being detected.

74. The method of any one of claims 33-73, wherein the method results in a CNV being identified.

75. The method of any one of claims 33-74, wherein obtaining the allele balance signal comprises correcting an original allele balance signal by performing the method of any one of claims 1-32.

76. The method of any one of the preceding claims, wherein the method comprises obtaining a signal indicative of ploidy status (e.g., the allele balance signal or depth of read signal) that is derived from a sample comprising a population of cells having different copy numbers for the chromosomal segment, optionally wherein some cells have an aneuploidy and others do not.

77. The method of any one of the preceding claims, wherein the method comprises obtaining a signal indicative of ploidy status (e.g., the allele balance signal or depth of read signal) that is derived from a sample comprising one or more tumor cells.

78. The method of claim 77, wherein the sample further comprises non-tumor cells.

79. The method of any one of the preceding claims, wherein the method comprises obtaining a signal indicative of ploidy status (e.g., the allele balance signal or depth of read signal) that is derived cell-free DNA, optionally wherein the cell-free DNA comprises cell-free fetal DNA (cffDNA) or circulating tumor DNA (ctDNA).

80. The method of any one of the preceding claims, wherein the method comprises obtaining a signal indicative of ploidy status (e.g., the allele balance signal or depth of read signal) that is derived from an embryo, optionally prior to implantation of the embryo into a womb.

81. The method of any one of the preceding claims, wherein the method comprises obtaining a signal indicative of ploidy status (e.g., the allele balance signal or depth of read signal) that is derived from a fetus.

82. A method of detecting chromosomal instability in tumor DNA, the method comprising:
determining a ploidy status according to any one of claims 32-81 for one or more chromosomal segments within a sample of genetic material that is at least partially derived from DNA originating from one or more cells known to be or suspected to be tumor cells, wherein identification of an aneuploidy status for the one or more chromosomal segments is used to indicate chromosomal instability of at least some tumor cells.

83. The method of claim 82, wherein the sample is from a subject diagnosed with or suspected of having cancer.

84. The method of claim 82 or 83, wherein the sample comprises circulating tumor DNA.

85. The method of any one of claims 82-84, wherein sequencing of normal tissue (e.g., germline tissue) from a subject from which the genetic material is obtained is used to establish a reference genetic code.

86. The method of any one of the claims 82-84, wherein sequencing on tumor tissue from a subject from which the genetic material is obtained is used to establish a reference genetic code.

87. The method of any one of claims 82-86, further comprising treating the one or more cells or a subject from which the genetic material is obtained for cancer based on whether chromosomal instability has been indicated.

88. The method of claim 87, wherein the treatment comprises administering poly ADP ribose polymerase (PARP) inhibitors to the one or more cells or subject if chromosomal instability is indicated.

89. The method of claim 87 or 88, wherein the treatment comprises administering platinum-based chemotherapeutics to the one or more cells or subject if chromosomal instability is indicated.

90. A method of detecting a de novo copy number variant (CNV) in a subject, the method comprising determining a ploidy status according to any one of claims 32-81 for a chromosomal segment, wherein the parents of the subject are euploid for the chromosomal segment, optionally wherein a de novo aneuploid (e.g., CNV) is identified in the chromosomal segment of the subject.

91. The method of claim 90, wherein the determination of ploidy status comprises comparing the ploidy status to a reference genetic code derived from sequencing performed on one or more genetic relatives of the subject.

92. The method of claim 91, wherein the one or more genetic relatives is a mother and/or a father.

93. The method of claim 91 or 92, wherein the sequencing is performed with a non-error-propagating technique to provide a plurality of reads according to any one of claims 1-32.

94. The method of any one of claims 91-93, wherein the sequencing is performed on cellular DNA.

95. The method of any one of claims 90-94, further comprising determining whether the mother or father of the subject is the source of an aneuploidy.

96. The method of any one of claims 90-95, wherein the subject is an embryo.

97. The method of claim 96, wherein the method comprises obtaining a signal indicative of ploidy status (e.g., the allele balance signal or depth of read signal) that is derived from one or more of an embryo biopsy, blastocele fluid, and cell culture medium.

98. The method of claim 97, wherein the signal indicative of ploidy status is obtained from cell-free DNA in the culture medium.

99. The method of any one of claims 96-98, further comprising selecting the embryo based on the absence or presence of an aneuploidy, optionally wherein the embryo is selected from a plurality of embryos.

100. The method of claim 99, further comprising using the selected embryo for in vitro fertilization (IVF).

101. The method of claim 99, further comprising disposing of the selected embryo.

102. The method of claim 99, further comprising freezing the selected embryo.

103. The method of any one of claims 90-94, wherein the subject is a fetus.

104. The method of claim 103, wherein the method comprises obtaining a signal indicative of ploidy status (e.g., the allele balance signal or depth of read signal) that is derived from cell-free fetal DNA (cffDNA).

105. The method of claim 103 or 104, further comprising treating the fetus and/or the mother based on the identified absence or presence of an aneuploidy (e.g., CNV).

106. The method of claim 105, wherein treatment comprises performing additional testing on the fetus, optionally wherein the additional testing comprises karyotyping.

107. The method of claim 105 or 106, wherein the treatment comprises terminating a pregnancy.

108. The method of any one of claims 105-107, wherein the treatment comprises administering a prenatal treatment to the fetus for a disease associated with the presence of a detected aneuploidy (e.g., CNV).

109. A method of screening a subject for a disease, the method comprising:
determining whether one or more genetic variants associated with the disease is present, wherein the one or more genetic variants comprises an aneuploidy (e.g., CNV) that was identified by performing the method of any one of claims 32-81 on one or more other subjects and/or an SNP that was present within a same haplotype block as the aneuploidy, optionally wherein the SNP is known to be associated with the disease.

110. The method of claim 109, wherein the one or more genetic variants comprises the aneuploidy.

111. The method of claim 109 or 110, wherein the one or more genetic variants comprises the SNP.

112. The method of any one of claims 109-111, wherein the CNV and SNP are in linkage disequilibrium.

113. The method of any one of claims 109-112, wherein determining whether the one or more genetic variants associated with the disease is present comprises performing sequencing on the subject, optionally wherein a portion of the genome comprising the one or more genetic variants is targeted (e.g., via a microarray).

114. The method of any one of claims 109-113, further comprising calculating a polygenic risk score (PRS) for the disease based at least in part on the one or more genetic variants.

115. The method of any one of claims 109-114, further comprising diagnosing the subject with a disease based at least in part on the presence or absence of the one or more genetic variants or on a PRS based at least in part on the one or more genetic variants.

116. The method of any one of claims 109-115, further comprising treating the subject based on the presence or absence of the one or more genetic variants.

117. A method of phasing a germline mosaic variant in a subject, the method
comprising:
obtaining a reference genetic code comprising two phase sets, each phase set having one or more variants of interest, optionally wherein the reference genetic code is at least partially phased;
obtaining a plurality of reads sequenced using a non-error-propagating technique, wherein each read comprises at least one of the one or more variants of interest;
determining the phase alignment of the two phase sets as being in phase or out of phase based on the plurality of reads; and
identifying a haplotype comprising a chromosomal segment exhibiting an aneuploidy (e.g., CNV) based on the determined phase alignment of the two phase sets.

118. The method of claim 117, wherein the subject is diagnosed or suspected as having a genetic disease or condition associated with the aneuploidy, optionally wherein the subject is diagnosed as having or suspected of having Noonan Syndrome or RASopathy.

119. The method of claim 117 or 118, further comprising screening gametes from the subject for the identified haplotype.

120. The method of claim 119, further comprising selecting a gamete not having the identified haplotype for in vitro fertilization.

121. The method of any one of claims 117-120, further comprising screening for the haplotype in an embryo during preimplantation genetic testing.

122. The method of claim 121, further comprising selecting an embryo based on the absence or presence of the aneuploidy, optionally wherein the embryo is selected from a plurality of embryos.

123. The method of claim 122, further comprising using the selected embryo in in vitro fertilization (IVF).

124. The method of claim 122, further comprising disposing of the selected embryo.

125. The method of claim 122, further comprising freezing the selected embryo.

126. The method of any one of claims 117-125, wherein the aneuploidy is identified by performing the method of any one claims 32-81.
